(19) United States
(12) Patent Application Publication
(10) Pub. No.: US 2004/0247661 A1
(43) Pub. Date: Dec. 9, 2004

(54) Liposomal vaccine

(76) Inventors: Dov Michaeli, Larkspur, CA (US); Stephen Grimes, Davis, CA (US); Yechezkel Barenholz, Jerusalem (IL); Simcha Even-Chen, Yavneh (IL)

Correspondence Address: WHITE & CASE LLP, PATENT DEPARTMENT, 1155 AVENUE OF THE AMERICAS, NEW YORK, NY, 10036, US

(21) Appl. No.: 10/759,832

(22) Filed: Jan. 15, 2004

Related U.S. Application Data
(63) Continuation-in-part of application No. 10/613,377, filed on Jul. 3, 2003.
(60) Provisional application No. 60/394,179, filed on Jul. 3, 2002.

(51) Int. Cl. A61K 38/22; A61K 9/127

(52) U.S. Cl. 424/450; 514/12

(57) ABSTRACT

The invention provides liposomal vehicles for encapsulating relatively high levels of water-soluble substances including immunogens directed against gastrin and gonadotropin releasing hormone. The liposome encapsulating large amounts of immunogens can be injected parentally to induce effective immune responses without exhibiting significant adverse tissue reactogenicity.

### CROSS-REFERENCE TO RELATED APPLICATIONS

This application is a continuation-in-part of U.S. Ser. No. 10/613,377 filed on Jul. 3, 2003, which claims the benefit, under 35 U.S.C.§119(e), of U.S. Provisional Application No. 60/394,179 filed on Jul. 3, 2002, each of which are incorporated herein by reference in their entireties.

### FIELD OF THE INVENTION

The invention relates to a liposome composition comprising a relatively high weight ratio of lipid material to encapsulated water-soluble compounds. In particular, the invention relates to injectable liposomal vaccines wherein large amounts of hydrophilic immunogens are efficiently and stably encapsulated in a plurality of liposomal vesicles for effective immunogenicity but with negligible tissue reactogenicity. The invention further relates to a process for the manufacture of the liposome vaccine composition.

### BACKGROUND OF THE INVENTION

Immunological neutralization or inhibition of hormones and their physiological effects can be useful in the therapeutic treatment of hormone dependent disorders and diseases by anti-hormone or anti-hormone receptor vaccination. For example, it has been widely accepted that reproductive and other hormones can act as growth factors that stimulate tumor growth including cancer of the breast, pancreas, lung, stomach, and colorectal system. Certain hormones which are not normally expressed and functional in healthy organs have been found to be active participants in the developing malignancy.

Although these hormones as self-antigens exhibit no inherent immunogenicity, treatment of disorders or diseases can be accomplished by the immunization of the subject patient or animal with an immunogenic carrier conjugated to an autologous target immunomimic peptide so as to induce an immune response producing anti-hormone or anti-hormone receptor antibodies. For example, U.S. Pat. Nos. 5,023,077; 5,468,494; 5,688,506; and 6,132,720 disclose immunogens and immunogenic compositions useful for neutralizing gastrin or gonadotropin releasing hormone activity.

It is further necessary to enhance the immunogenicity of such conjugates in order to render them useful in the clinic. One approach is to formulate them further with an oily vehicle to form emulsions for slow release. Water-soluble vaccines include anti-hormone or anti-hormone receptor targeted immunogens. Injectable immunogens are usually delivered in the form of a water-in-oil emulsion. These vaccine emulsions are limited as to how much dosage can be administered due to the inherent inflammatory tissue reactogenicity that develops at the injection site after immunization. Thus, this tendency to react locally resulted in some cases in administration of sub-optimal levels of immunogens.

Water-in-oil emulsions are composed of tiny doplets of water dispersed in a continuous oil phase (mineral, squalene or squalane or mixtures thereof). Metabolizable oils such as squalene or squalane have desirable safety aspects in that they are more patient amenable than Freund's Adjuvants which are unacceptable for human treatment. The prior art immunogen compositions, e.g., against gastrin or gonadotropin releasing hormone (GnRH), are formulated as water-in-oil emulsions that significantly enhance immune response. However, the immunization with vaccine-emulsion formulations potentially induces injection site reactions that may be acceptable in the treatment of life threatening diseases, but are discomforting in other conditions and, therefore, undesirable or even unacceptable. Hence, other modes of delivery of antigens have been explored. For example, liposomal influenza vaccines have been disclosed in U.S. Pat. No. 5,919,480 to Kedar, et al. wherein liposomes are used to encapsulate influenza subunit antigens and serve as vesicle-type delivery vehicles.

Although liposomes have good targeting potential and provide a basic formulation for incorporating hydrophilic and lipophilic immunomodulators, they are difficult to formulate so as to encapsulate sufficiently large amounts of immunogen, and often need help from soluble immunomodulators to be effective. J. C. Cox et al. “Adjuvants—a classification and review of their modes of action” in Vaccine 1997 Vol. 15 (13): 248-256.

The protein carrier capacity of the liposomal preparation has certain limitations. For example, the larger the proportion of protein in the liposomal compartment, the greater is the viscosity of liposome preparation. This viscosity can increase to a level so as to present a barrier against its use as an injectable vaccine. In fact, the highest encapsulation level by liposomes as injectables achieved thus far is about 30%. G. Gregoriadis (ed.), Liposome Technology, vol. 1, 2nd ed., CRC Press, Boca Raton, Fla. 1993, pp.527-616. Moreover, since the encapsulation efficiency of hydrophilic molecules within a liposome is especially low, liposome formulations have generally been better suited for amphipathic immunogens.

It has also been found that liposomes as vaccine delivery vehicles of hydrophilic antigen with low immunogenicity have required relatively large amounts of vaccine dosages. To date, such desired liposome-encapsulated immunogenic dose levels have not been attained, which is also in part due to limitations placed on the injection volume.

### SUMMARY OF THE INVENTION

The invention relates to an injectable liposomal composition for delivery of large amounts of a water-soluble substance. The composition comprises a plurality of liposomal vesicles having a high weight ratio of a lipid to an encapsulated water-soluble substance which is distributed over the plurality of liposomal vesicles. The weight ratio of lipid to encapsulated substance ranges from about 50 to 1000. This arrangement advantageously permits a high efficiency of encapsulation, for example, more than about 50% and in accordance with some embodiments, more than about 80%.

The liposomal vesicles of the present invention can be multilamellar vesicles (MLV). The liposome comprises liposome-forming lipids having a hydrophilic tail portion and a polar or chemically reactive portion which in turn comprises an acid, alcohol, aldehyde, amine or ester. The liposomes may be further characterized by hydrocarbon chains or steroid tail group and a polar head group. The liposome-forming lipids comprise a phospholipid. Examples of suitable phospholipids include, but are not limited to phosphatidic acid, phosphatidyl choline, phosphatidyl ethanolamine, phosphatidyl glycerol, phosphatidyl inositol and sphingomyelin.

The water-soluble substances that can be encapsulated in the liposomes of the present invention include proteins, proteoglycans and carbohydrates. In some embodiments, the water-soluble substance comprises more than one compound.

The water-soluble substance to be encapsulated may also be a vaccine including, but not limited to a vaccine against a hormone or a hormone cognate receptor. In accordance with specific embodiments of the invention, the vaccine comprises at least one hormone-immunomimic peptide or hormone receptor-immunomimic peptide conjugated to an immunogenic hydrophilic carrier protein. For example, the immunomimic peptide is a synthetic sequence selected form the group consisting of gastrin G-17, gastrin G-34, GnRH and hCG. Specifically, the synthetic gastrin G-17 has the sequence of SEQ ID NO: 1. Fragments of gastrin G-17 useful for the practice of the present invention include those of sequences shown in SEQ ID NOS: 3-8. The synthetic G34 peptide has the sequence of SEQ ID NO: 12. The synthetic GnRH immunomimic peptide has the sequence of SEQ ID NO: 15. The synthetic hCG immunomimic peptide sequence has the sequence of SEQ ID NO: 16. Spacer peptides useful for the practice of the present invention include, but are not limited to SEQ ID NOS: 9, 10 and 11.

In accordance with certain embodiments of the invention, the liposomes encapsulate, either separately or together, a water-soluble immunogen and a water-soluble high molecular weight immunomodulatory substance or, alternatively, a low molecular weight immunomodulatory substance. The high molecular weight immunomodulatory substance may be comprised of cytokines. Examples of the low molecular weight immunomodulatory substance include, but are not limited to, nor MDP, threonyl MDP, murabutide, N-acetylglucosaminyl-MDP and murametide.

The present invention is also directed to pharmaceutical formulations comprising the liposomal compositions and a pharmaceutically acceptable carrier. The pharmaceutical formulations of the present invention can include low viscosity liposomal compositions, as disclosed and claimed herein, and a pharmaceutically acceptable carrier. The pharmaceutical formulations of the invention may be administered to patients in need thereof as part of a therapeutic regimen in the treatment of a disorder or disease.

One example of such a pharmaceutical formulation is an aseptic composition comprising an injectable aqueous suspension of the low viscosity liposomal composition as disclosed and claimed herein. Since large amounts of protein can be stored in the liposomes, these large amounts of protein are delivered to provide a more immunogenic dose while keeping the viscosity suitable for injection and maintaining an acceptable dose volume. Thus, the invention provides for effective immunization without requiring potentially toxic adjuvants and immunomodifying additives. Furthermore, there is an advantageous reduction in tissue reactogenicity.

The invention also relates to a method of producing a liposomal vaccine comprising the steps of preparing phospholipid multilamellar vesicles and encapsulating water-soluble immunogen and/or immunomodulating substances whereby the liposomes have a high lipid to protein ratio.

### BRIEF DESCRIPTION OF THE DRAWINGS

FIG. 1 is an electron micrograph of a liposomal DMPC+G17DT conjugate composition, wherein the ratio of lipid to protein is 500:1.

FIG. 2 is an electron micrograph of a liposomal DMPC+G17DT conjugate composition and nor-MDP additive wherein the lipid:protein ratio is 500:1.

FIG. 3 is an electron micrograph of a liposomal DMPC/DMPG+G17DT conjugate composition wherein the lipid/protein ratio is 500:1.

FIG. 4 is an electron micrograph of a liposomal DMPC/DMPG+G17DT+nor-MDP, wherein the liquid/protein ratio is 500:1.

FIG. 5 is a graph of the mean anti-gastrin G17 antibody titers induced over time by vaccination comparing the control 100 μg G17DT conjugate alone or 100 μg G17DT injectable emulsion and 1.5 mg or 3 mg G17DT in liposomes (0 cu IL-2), and 1.5 mg or 3 mg G17DT in PBS, with 1.5 mg or 3 mg G17DT in liposomes plus 1000 cu IL-2 to 100,000 cu IL-2.

FIG. 6. is a graph of median anti-gastrin G17 antibody titers induced over time by vaccination with the above-identified compositions.

FIG. 7. is a graph of mean anti-GnRH antibody titers induced over time by vaccination with the control 100 μg GnRHDT conjugate or as an emulsion and control 1.5 mg or 3 mg GnRH—DT liposomes (0 cu IL-2), and 1.5 mg or 3 mg GnRHDT in PBS-solution, with 1.5 mg or 3 mg GnRHDT liposomes plus 1000 cu IL-2 to 100,000 cu IL-2.

FIG. 8. is a graph of the median anti-GnRH antibody titers induced over time by vaccination with the immunogens described above.

FIG. 9. is a graph of the mean anti-G17 rabbit antibody titer responsive to high dose G17DT liposomes reconstituted with 5% EtOH or water.

FIG. 10. is a graph of the median anti-G17DT rabbit antibody titers responsive to high dose G17DT liposomes reconstituted with 5% EtOH or water.

### DETAILED DESCRIPTION OF THE INVENTION

The following terms are described as to meaning and use in the context of the present invention.

Liposome-forming lipids or vesicle-forming lipids refer to amphipathic lipids characterized by hydrophobic and polar head group moieties, which can spontaneously form bilayer vesicles in water. Specifically, liposome-forming lipids are stably incorporated in lipid bilayers such that the hydrophobic moiety is in contact with the interior region of the vesicle membrane while the polar head group moiety is oriented to the exterior, polar surface of the vesicle membrane.

The term “separately encapsulated” as used herein refers to liposome-encapsulated ingredients, wherein e.g., an antigen and a cytokine are separately encapsulated in different liposomal preparations.

In contrast, “co-encapsulated” ingredients are understood as a liposomal preparation containing a combination of more than one antigen or product, as e.g., antigen and immunostimulating agents.

As stated above, the inventive liposomes of the present invention are suitable for encapsulating water-soluble substances, such as hydrophilic proteins and also low molecular weight compounds, so as to effect distribution of large amounts of substance over a great plurality of lipid vesicles, usually ranging from 0. 1-10 μm in size.

More specifically, the liposome-encapsulated water-soluble compounds can include vaccine constructs comprising immunomimic and/or immunogenic moieties. The constructs can comprise conjugates of immunogenic carrier proteins and target-immunomimic peptides. The carrier protein may include immunogenic fragments as carrier.

The term “injectable composition” defines a liposomal composition that possesses a viscosity low enough to permit parenteral injection by, e.g., a hypodermic needle.

As used herein “efficiency of encapsulation” is defined as the proportion or percentage of protein (or other antigen) that is associated with (i.e. taken into and/or bound to the surface of) liposomes relative to the total amount of protein (or other antigen) added to the system. The remaining protein (or other antigen) is not associated with liposomes and remains free in the aqueous vehicle.

Liposome formulations generally have been regarded as most suited for encapsulating amphipathic substances. Unexpectedly, liposomes prepared in accordance with the invention with high lipid to protein weight ratio conditions are capable of encapsulating large amounts so that, for example, at least 50% of water-soluble substances are distributed in large numbers of lipid vesicles. This was accomplished without allowing the preparation to become too viscous for injection. Furthermore, the high lipid-to-protein ratio (HLPR) of the liposomal preparation according to the invention serve to significantly reduce or even eliminate reactogenicity while increasing immunogenicity to clinically effective levels by the substantially increased doses of liposome encapsulated immunogen. Thus, the liposomes of this invention are much better tolerated than the conventional water-in-oil emulsions while still achieving in vivo effective immune responses.

The high lipid to protein ratios of liposomal preparations reduce reactogenicity of an anti-hormonal vaccine while multilamellar liposomal vaccines against autologous hormones do not induce sufficient antibody titers when the liposomes were formulated with the low doses of emulsified immunogen according to the prior art. In fact, previous attempts by others to increase the content of hydrophilic immunogens in liposomes were also unsuccessful as the efficiency of encapsulation of hydrophilic molecules was generally poor.

The liposomal vesicles of the invention comprise lipid bilayer membranes formed in water from lipids arraying hydrophobic tail group moieties and polar head group moieties. The hydrophobic tail moieties include saturated hydrocarbon chains and steroid groups, while the polar head groups comprise chemically reactive groups such as acid, alcohol, aldehyde, amine, and ester moieties. For example, such vesicle forming lipids with acid head groups include the phospholipid group. According to the invention, the phospholipids include, but are not limited to, phophatidic acid (PA), phosphatidyl choline (PC), phosphatidyl ethanolamine (PE), phosphatidyl glycerol (PG), phosphatidyl inositol (PI) and sphingomyelin (SM).

The encapsulated water-soluble immunogens are can comprise target antigen-immunomimic peptides linked to an immunogenic water-soluble carrier protein. Since the hydrophilic portion of the water-soluble immunogenic carrier protein predominates, it substantially affects the overall water-soluble character of the entire immunogenic construct.

Another embodiment of the invention comprises hydrophilic immunogenic carrier proteins comprising Diphtheria toxoid (DT), Tetanus toxoid (TT), horseshoe crab hemocyanin, Keyhole limpet hemocyanin (KLH), bovine serum albumin (BSA), or the polysaccharide dextran; or the immunogenically active components of these respective carrier entities.

The liposomal vaccine composition of the present invention can comprise a large amount of water-soluble vaccine stably encapsulated in a large plurality of liposomes which are suspended in an aqueous carrier, and wherein each liposomal particle is immunogenic so as to effect a sustained, clinically significant immune response.

The liposomal vaccine suspension comprising the immunogen and/or immunomodulatory substances targeted against autologous antigens is suitable for administration to a patient for the purpose of treatment against autologous target related diseases or disorders.

The liposomal immunogen may be administered to a patient under such treatment by the parenteral, nasal, rectal, or vaginal route. The parenteral administration includes intravenous, intramuscular, subcutaneous, intradermal and intraperitoneal injections.

For example, immunization by injectable liposomal vaccine can be directed against reproductive hormones so as to interrupt conception. Pursuant to another example, the immunization with liposomal anti-GnRH or anti-hCG vaccine as described below can effect immune response so as to prevent pregnancy.

An advantageous embodiment of the injectable suspension of high lipid to protein weight ratio vesicles (HLPR) provides high doses of encapsulated immunogenic conjugate of Diphtheria toxoid protein (DT) in a large number of suitably sized lipid vesicles which can be safely injected for immunization against self-antigens. Such autologous immunization targets include normal hormones and similar factors and their cognate receptors involved in stimulating (either directly or indirectly) tumor growth in various gastrointestinal or reproductive systems, or in promoting metastatic cancers of colorectal, breast, or prostate origin.

Thus, the invention comprises an injectable aqueous suspension of liposomal vesicles encapsulating an anti-gastrin immunogen construct. Another embodiment of the invention comprises an injectable aqueous suspension of liposomal vesicles encapsulating an anti-GnRH immunogenic construct. The invention also provides a human chorionic gonadotrophic (hCG) immuno-contraceptive vaccine encapsulated in the HLPR liposomes. Accordingly, an embodiment provides the liposomal anti-hCG vaccination suitable as contraceptive, entailing reduced tissue reactogenicity while providing clinically efficacious doses of immunogen.

In addition, certain hormones or growth factors are only partially processed in cancer to immature forms which have been found to exhibit autocrine and/or paracrine activities in tumors. For example, it is known that the hormone, gastrin, that is both amidated gastrin-17 (G17), pGlu-Gly-Pro-Trp-Leu-Glu-Glu-Glu-Glu-Glu-Ala-Tyr-Gly-Trp-Met-Asp-Phe-NH₂ (SEQ ID NO: 1 in the Sequence Listing), and the precursor form glycine-extended gastrin-17 (GlyG17), pGlu-Gly-Pro-Trp-Leu-Glu-Glu-Glu-Glu-Glu-Ala-Tyr-Gly-Trp-Met-Asp-Phe-Gly (SEQ ID NO: 2) can stimulate both gastrointestinal (GI) tumors and also non-GI related tumors such as the tumors of thyroid cancer and lung cancer.

An anti-gastrin directed embodiment of the invention comprises an injectable aqueous suspension of the large number of small liposomal vesicles of high lipid-to-protein ratio encapsulating large amounts of hydrophilic anti-gastrin G17 immunogenic constructs which may contain a G17-aminoterminal epitope immunomimic peptide of various lengths ranging such as for instance, amino acid positions 1-5, 1-6, 1-7, 1-8, 1-9, or 1-10 (SEQ ID NO: 3, 4, 5, 6, 7, or 8 respectively), linked at its C-terminus either through a six-residue peptide spacer (e.g. SEQ ID NO: 9), a seven-residue peptide spacer (e.g. SEQ ID NO: 10), or an eight-residue peptide spacer (e.g. SEQ ID NO: 11) to the carrier protein.

Another embodiment of the invention provides a liposomal immunogen directed against the N-terminal peptide sequence 1-22 of the gastrin hormone, G34 (SEQ ID NO: 12) which is useful for the immunogenic control or inhibition of gastrin and secretion.

In this context, an embodiment provides an immunomimic synthetic peptide, pGlu-Leu-Gly-Pro-Gln-Gly-Ser-Ser-Pro-Pro-Pro-Pro-Cys or Cys-Pro-Pro-Pro-Pro-Ser-Ser-Glu-Leu-Gly-Pro-Gln-Gly (SEQ ID NO: 13 and 14, respectively), linking the G34 (1-6 aa) fragment with the spacer peptide, e.g. Ser-Ser-Pro-Pro-Pro-Pro-Cys (SEQ ID NO: 11) either at the C-terminal or the N-terminal end whereby the immunomimic peptide is conjugated at the Cys residue to a suitable immunogenic carrier protein.

In addition, the mammalian reproductive hormone, Gonadotropin Releasing Hormone (GnRH), pGlu-His-Trp-Ser-Tyr-Gly-Leu-Arg-Pro-Gly-NH₂ (SEQ ID NO: 15), has been implicated in the growth of cancer in both the male and the female reproductive systems.

An embodiment of the injectable suspension of vesicle-type liposomes having a high lipid to protein ratio with encapsulated immunogen provides a spacer peptide linking the immunogenic carrier to the hormone-immunomimicking synthetic peptide, such as, e.g., Diphtheria toxoid conjugated to a peptide analog of gastrin-17, or a gonadotropin releasing hormone immunomimic analog or fragment thereof.

The appropriate sequences are selected for conjugation to Diphtheria toxoid or Tetanus toxoid according to the methods disclosed in U.S. Pat. No. 4,767,842, which description (i.e. the hCG Structure II) is entirely incorporated in this application by reference. For an hCG-immunogenic construct, an hCG immunomimicking synthetic peptide can be linked to the immunogenic carrier, DT. Other immunogenic proteins, such as those set forth above, would also be useful carriers of the hCG peptide construct.

An embodiment includes a hCG fragment corresponding to a portion of the 111-145 amino acid sequence of the beta subunit of hCG (SEQ ID NO: 16 in the Sequence Listing) (“Structure II” recited in U.S. Pat. No. 4,767,842) which is not common to LH (Luteinizing Hormone) and, therefore, would not produce LH cross-reactive antibodies. Another embodiment of the invention provides a hCG-immunomimic synthetic peptide including an eight-member peptide spacer (SEQ ID NO: 11) at the N-terminus of the hCG beta submit, ranging from position 138-145 at the C-terminal end of hCG (SEQ ID NO: 17), linked to DT. Other spacer peptides (SEQ ID NO: 8 or 9) are also useful for an anti-hCG immunogen construct.

A pharmaceutical embodiment of the invention provides an injectable suspension of liposomal vesicles encapsulating an anti-hCG immunogenic construct at a high lipid to protein weight ratio, and pharmaceutically acceptable carrier.

An embodiment of the invention provides a method for producing a large number of an injectable liposomal preparation encapsulating a relatively large amount of vaccine in a great number of lipid particles. The method can include chemically stabilized liposome encapsulation of immunogens directed against cancer growth-promoting hormones and their cognate receptors.

A further embodiment of the invention provides the method of producing numerous lipid vesicles for loading large amounts of water-soluble immunogens achieving a high lipid-to-protein weight ratio. Such a method can encapsulate and adsorb hormone immunomimic peptides such as G17 or GnRH, conjugated to a hydrophilic immunogenic carrier protein or fragment thereof.

According to the invention, the size of a liposomal vesicle may range from 0.1 μm to about 10 μm. Furthermore, the liposomal suspension can provide an encapsulated vaccine load of about 50 ug to about 5 mg, or more preferably approximately 0.3 mg to approximately 5 mg protein at a lipid-to-protein ratio ranging from about 50:1 to about 1000:1. Preferably, the lipid-to-protein ratio is in the range of about 100:1 to about 500:1.

It will be recognized that the optimal ratio of protein to lipid may differ for different proteins, and this optimal ratio may be readily established for each different protein by methods, such as those described herein, that are well known to those skilled in the art. Similarly, other forms of vaccine antigen, such as intact, fractionated or aggregated forms of peptides, proteins, viruses, bacteria, or fungi, as well as hormones or drugs, may differ in the optimal immunogen:lipid ratio when delivered in liposomes of this invention. The optimal immunogen:lipid ratio for each of these formulations can be readily determined by well known methods that are routinely used by those of skill in the art.

The liposome of the invention can be prepared to co-encapsulate or separately encapsulate, at least one high molecular weight or low molecular weight immunomodulatory adjuvant. High molecular weight immunomodulatory adjuvants include, but are not limited to, isolated cytokines or microparticles of non-ionic block copolymer. An effective dose of encapsulated cytokines comprises interleukins such as IL-1, IL-2, IL-4, IL-6, IL-7, IL-12, IL-15, or IFN-gamma, muramyl dipeptide (MDP) or hydrophilic derivatives thereof, such as nor-MDP, threonyl MDP, murabutide, N-acetylglucosaminyl-MDP, and murametide, and, furthermore, the lipid A derivative, 4′-monophosphoryl lipid A (MPL), the triterpenoid mixture Q521 or ISCOPREP™ 703 (a defined Saponin), CpG-oligodeoxynucleotides and Tomatine (a glycoalkaloid saponin, C₅₀H₃NO₂₁; Sigma). The immunomodulatory substance of the liposome preparations of the present invention, co-encapsulated or encapsulated separately, can include IL-2, ranging from about 10 c.u. to about 100,000 c.u. The liposomal composition also provides combinations of immunogenicity-enhancing additives, such as, e.g., a combination of IL-2 and a non-ionic block polymer.

The present method of immunization with low tissue reactogenicity comprises administration of a suspension of liposomes encapsulating water-soluble protein compounds at a high lipid to protein weight ratio. The lipid vesicle encapsulated protein comprises an anti-hormone immunogen or anti-hormone receptor immunogen as well as an immunomodulating compound which can be separately encapsulated or co-encapsulated in the same preparation which can be administered by the intramuscular, subcutaneous, intranasal or rectal route.

Without engaging in undue theoretical speculation, it is presently presumed that the invention provides a transport vehicle wherein the encapsulated protein is located in the lipid vesicle so as to afford two kinds of delivery modes. Specifically, the delivery modes include both rapid delivery which takes place by releasing the adsorbed antigen from the exterior surface of the vesicle, as well as slow, more prolonged release of the antigen component from the complete enclosure by the membrane system of each lipid vesicle.

Another aspect of the invention provides a method of prolonged immunocontraception with effectively slow release delivery of liposome internalized immunogen, without the need for frequent booster immunization.

The invention also provides methods for producing liposomes of high lipid to protein ratios which are able to encapsulate relatively large amounts of water-soluble antigen.

The immunogen constructs are prepared according to the methods described in the co-assigned U.S. Pat. Nos. 5,023,077; 5,468,494; 5,688,506; 5,698,201 and 6,359,114. In principle, the immunogenic carrier protein or immunogenic fragment thereof is conjugated either directly or indirectly, through a suitable immunologically inert spacer peptide to a peptide of suitable length, which peptide immunomimics the target hormone or receptor moiety so as to generate the specific anti-hormone or hormone receptor antibodies capable of neutralizing or inhibiting the hormone directed physiological effect. The usual molar ratio of immunomimic peptide to immunogenic carrier protein ranges from 1 through 40 moles wherein the unit carrier is placed at 10⁵ MW.

The following examples illustrate the advantageous aspects of the invention, which is, however, not limited to the described water-soluble compounds, including peptide hormones or hormone receptors as targets for immunizations. Co-assigned U.S. Pat. Nos. 5,023,077, and 5,468,494 disclose immunogens for neutralizing gastrin and U.S. Pat. No. 5,688,506 discloses GnRH activity in humans and other mammalian subjects. The entire disclosure of these patents is incorporated herein by reference. U.S. Pat. No. 5,698,201 discloses the production of human chorionic gonadotropin (hCG) immunogens, which entire method is incorporated herein by reference. Moreover, the anti-gastrin immunogenic conjugate has been selected as a candidate for immunization treatment against gastrointestinal malignancy. (See review by Watson et al. Exp. Opin. Biol. Ther 2001, 1 (2): 309-317).

The various liposomal immunogens of the invention may comprise synthetic immunomimicking hormone peptide fragments, such as, e.g., gastrin G-17, (SEQ ID NO: 7); or human GnRH, (SEQ ID NO: 15).

The gastrin immunomimic peptide may comprise a sequence length of 5 amino acids or greater, as for example, N-terminal 1-5, 1-6, 1-7, 1-8 or 1-9 amino acid sequences of the various G17 (SEQ ID NO: 3, 4, 5, 6, 7, or 8) hormone immunogenic constructs with a C-terminally attached spacer, such as for instance, SSPPPPC.

The G17DT construct as encapsulated by processes described in the Examples 1 and 2 is a gastrin immunogen includes a G17 immunomimic nonapeptide derived from the aminoterminal portion (1-9) of human G17 which is extended by a spacer element comprising an additional seven amino acids at its C-terminus. The resulting hexadecapeptide pGlu-Gly-Pro-Trp-Leu-Glu-Glu-Glu-Glu-Glu-Ser-Ser-Pro-Pro-Pro-Pro-Cys (SEQ ID NO: 18) is covalently linked to the carrier molecule Diphtheria toxoid (DT) through the sulfhydryl group on the terminal cysteine residue by reacting with heterobifunctional linker molecule to the ∈-amino groups of the lysine residues present on the carrier protein.

The amino acid sequence 1-10 of GnRH may be selected as a GnRH immunogen. The immunogen may also comprise a peptide spacer linking the carrier to the immunomimicking peptide, such as, e.g., in international amino acid terminology, RPPPPC (SEQ ID NO: 9), SSPPPPC (SEQ ID NO: 10), but are not limited to these. Another suitable spacer is found in SPPPPPPC (SEQ ID NO: 11). The GnRH immunomimicking synthetic peptide is linked covalently through a spacer peptide to the carrier by reacting the terminal cysteine (C) by disulfide bonding.

The GnRH conjugate encapsulated in the liposomes described in Example 4 is also identified as “D17DT” which is the septadecapeptide, Cys-Pro-Pro-Pro-Pro-Ser-Ser-Glu-His-Trp-Ser-Tyr-Gly-Leu-Arg-Pro-Gly-NH₂ (SEQ ID NO: 19), comprising the aminoterminal GnRH immunomimic sequence which is extended the spacer peptide which is linked at its C-terminus through a heterobifunctional reagent to the ∈-amino groups of the lysine residues present in the carrier protein, i.e., DT.

G17-Diphtheria toxoid (G17DT) conjugate immunogen is constructed to induce antibodies that specifically neutralize human gastrin G17 (hG17). The immunogen can comprise peptides bearing a hG17 epitope that are covalently linked to a hydrophilic immunogenic carrier, such as Diphtheria toxoid (DT). G17-immunomimic peptides comprise fragments extending from the N-terminal end of G17 up to amino acid number 5 through 12. These G17 peptide fragments are optionally linked to a spacer such as the SSPPPPC peptide, and to an immunogenic hydrophilic carrier, such as DT. Similarly, immunogens can be constructed with immunomimics of the C-terminal sequence portion of G17 or Gly-extended G17. The immunogenic conjugates, which can be dissolved in an aqueous phase, is designed to elicit anti-gastrin antibody production in vivo. Nevertheless, the induction of effective levels of anti-hG17 antibodies with a practical immunization regimen requires that the immunogenicity of the conjugate be enhanced by inclusion of an immunopotentiating adjuvant.

The synthetic hCG immunogen can include Cys-Pro-Pro-Pro-Pro-Ser-Ser-Ser-Asp-Thr-Pro-Ile-Leu-Pro-Gln (a 138-145 aa C-terminal peptide sequence; SEQ ID NO: 20).

The invention provides methods for producing injectable liposome-encapsulated vaccines containing large amounts of immunizing protein eliciting high titer antisera, unrestricted with regard to tissue reaction at the injection site the liposomes, thus achieving an advantageous ratio of high antibody titer in relation to low or negligible reactogenicity. The following detailed description and examples disclose the composition of the multilamellar liposomal vesicles of the invention, and especially the production of the compositions of liposomes which are suitable to encapsulate hydrophilic immunogens.

As shown in the examples set forth below, it has been found that encapsulated doses of immunogen in amounts as high as 1.5 or 3.0 mg in multilamellar liposomes are significantly less irritating to the local tissue than, for example, the much lower dose of 100 μg immunogen in the water-in-oil emulsion formulation.

A variety of liposomal vesicle-forming lipids can be used for forming liposomal compositions, according to methods that are well known in the art. The relevant methods and materials for the liposomal vesicle as disclosed in U.S. Pat. No. 5,919,480, are incorporated herewith by reference, and further described below. The lipids or oily vesicle forming substances of the invention allow long-term storage of the liposome-capsulated antigen and adjuvants and effective release of these components upon administration. Representative lipids include, but are not limited to, dimyristoyl phosphatidylcholine (DMPC), dimyristoyl phosphatidylglycerol (DMPG), cholesterol, 1,2-distearoyl-3-trimethylammonium propane (DSTAP), 1,2-dimyristoyl-3-trimethylammonium propane (DMTAP), and combinations thereof, such as DMPC/cholesterol, DMPC/DMPG, DMPC/DMPG/cholesterol, DMPC/DMTAP, and DMPG/DMTAP/cholesterol. Liposomal compositions of the inventions may contain 10-100 mole percent DMPC. In a preferred embodiment the liposomal compositions of the present invention includes at least 70 mole percent DMPC. Particularly useful compositions provide mixtures of 9:1 (mol/mol) DMPC/DMPG and DMPC alone.

The liposomes of the invention can also include large lipid vesicles, as described below, having a mean diameter of approximately 0.25 μm to approximately 5.0 μm.

The invention provides an immune response enhancing compound which may be coencapsulated with targeting immunogenic liposome, or alternatively encapsulated in an appropriately constructed multilamellar liposome for injection at a separate or very nearly the same locations as the immunogen.

The liposomal immunogenic composition can also contain immunostimulating cytokines, also identified as interleukins. The cytokine additive includes a selection of an interleukins, such as Il-1, Il-2, Il-4, Il-6, Il-7, Il-12, Il-15, IFN-gamma, and a granulomacrophage colony stimulating factor (GM-CSF) or combination thereof. For example, the immunomodulatory agents Il-2 and GM-CSF may be combined for the immunizing treatment via liposomal delivery.

The cytokines can be included as high molecular weight adjuvants which are glycoproteins of about 20 KD (KD=kilodaltons) or more. Cytokines have different targets toward effecting an enhanced immune response, IL-1 enhances T and B cell maturation, IL-2 enhances T and B lymphocyte and phagocyte upregulation, IL-4 enhances B-cell upregulation, IFN-gamma enhances B cell and macrophage upregulation and enhances MHC expression, and GM-CSF represents a co-migratory signal for dendritic cells (DCs).

The liposomal vaccine of the invention may include liposome-encapsulated adjuvants, which are administered individually or together with the immunogenic conjugates to the treated subject. For example, the immunomodulatory adjuvant comprises a low molecular weight compound, such as the nor-muramyl dipeptide (nor-MDP). The dosage can be any effective and acceptable amount, which can range from 1 through 50 μg nor-MDP per dose.

Nor-MDP is a less toxic hydrophilic derivative of N-acetylmuramyl-L-alanyl-D-isoglutamine, which is an adjuvant-active component of a peptidoglycan extract of Mycobacteria. Other hydrophilic derivatives include threonyl MDP, murabutide, N-acetylglucosaminyl-MDP and murametide. Nor-MDP tends to stimulate Th2 lymphocytes. The lipophilic derivative MTP-E tends to stimulate Th-1 lymphocytes.

Liposome formulations can incorporate various combinations of low molecular weight immunomodulatory molecules, including MPL, lipophilic MDP or hydrophilic nor-MDP, defined saponin Q521, ISCOPREP™ 703, or Quil A, and CpG-oligodeoxynucleotides. Liposome-suitable adjuvant for human vaccine may also include 4′-monophosphoryl lipid A (MPL) derived from Lipid A. Tomatine, a saponin, is a naturally derived glycoalkaloid having immunopotentiating activity (Sigma).

Other strong immunostimulatory adjuvants can include the non-ionic block polymers located in the aqueous phase of standard water-in-oil emulsions which have been observed as eliciting an apparent level of immunity sustained for at least four months without inducing an unacceptable level of local irritant reactivity of the injection site. Synthetic polymers such as polylactide coglycolide (PLG), Calcium salts, collagens, Calcium or Sodium hyaluronate, polyethylene glycol (PEG) or other gel forming substances can also be added in the form of microspheres which degrade yielding a pulsed delivery of immunogen and immunostimulating adjuvant. Such release control can extend the immunization effect for several months.

Preparation of Liposomes and Liposomal Compositions:

The methods of preparing liposomal suspensions containing water-soluble encapsulated agents in accordance with the invention, and methods of incorporating additional components into the liposomes are described below.

Liposomes may be prepared by a variety of techniques. To form multilamellar vesicles (MLV), a mixture of vesicle-forming lipids is dissolved in a suitable organic solvent (or solvent mixtures) and evaporated in a vessel to form a thin film, which is then hydrated by an aqueous medium to form lipid vesicles, typically in sizes ranging from about 0.1 to about 10 μm. Tert-butanol (TB) is a particularly suitable solvent for the process, followed by lyophilization (MLV prepared using this solvent are termed TB-MLV). The lyophilized MLV preparation can be resolubilized as an aqueous suspension. The MLV suspension can then be selectively downsized to a desired vesicle size range of 1 micron or less by extruding aqueous suspension through a polycarbonate membrane having a select uniform pore size, typically 0.05 to 1.0 microns.

Vesicle-forming lipids according to the invention contain hydrophobic chains and polar head group moieties so as to be able to form bilayered vesicles in water. For example, phospholipids may spontaneously form vesicles in an aqueous environment or are stably incorporated into lipid bilayer membranes with the hydrophobic portion of the lipid molecule in the interior and the polar head group portion of the lipid molecule in the hydrophilic, external surface of the bilayer vesicle. The lipid bilayer membrane of the liposomal vesicle is designed to hold the hydrophilic immunogen within and on the lipoid membrane vesicle enclosure.

Vesicle-forming lipids may include hydrocarbon chains, a steroid group, or a chemically reactive group, such as acid, alcohol, aldehyde, amine or ester, as a polar head group. The phospholipids include vesicle forming combinations of phosphatidic acid (PA), phosphatidyl choline (PC), phosphatidyl ethanolamine (PE), phosphatidyl glycerol, phosphatidyl inositol (PI), and sphingomyelin (SM) which generally comprise two hydrocarbon chains of about 14-22 carbons at varying degrees of unsaturation. Lipopolymers can be added to stabilize the lipid content of the vesicles,. Furthermore, vesicles can be formed from glycolipids, including cerebrosides and gangliosides, as well as sterols (i.e. cholesterol). Synthetic membrane forming phosphatidyl derivative compounds containing dihexadecyl, dioleoyl, dilauryl, dimyristoyl, or dipalmitoyl groups are also available (Calbiochem), including dimyristoyl phosphatidyl choline or dimyristoyl phosphatidyl glycerol which can be taken as a mixture, with and without lipid membrane stabilizing additives.

While immunogenic liposome compositions conventionally utilize low amounts of highly antigenic viral particles, the very low or negligible antigenicity of an organism's own, i.e. autologous, hormones or hormone receptors not only requires a highly immunogenic carrier protein, such as e.g. Diphtheria toxoid or Tetanus toxoid for vaccination, but hormone immunogen liposomes have also been found to require considerably larger amounts of the autologous antigen distributed over a large number of encapsulating liposomes so as to maintain chemical stability and favorable delivery conditions while preventing undesirable degrees of reactogenicity. In addition to the aforementioned immunogens, the liposomes of the invention would be suitable for delivery of other water-soluble substances, including hormones, growth factors, cofactors, or adjuvants which can be modified for increased immunogenicity.

Various methods are available for encapsulating other or additional agents in the liposomes. For example, in the reverse phase evaporation method (Szoka, U.S. Pat. No. 4,235,871) a non-aqueous solution of vesicle-forming lipids is dispersed with a smaller volume of an aqueous medium to form a water-in-oil emulsion. Thus, for encapsulation the active ingredients or agents are included either in the lipid solution, in the case of a lipophilic agent, or in the aqueous medium, as in the case of a water-soluble agent. After removal of the lipid solvent, the resulting gel is converted to liposomes. These reverse phase evaporation vesicles (REVs) have typical average sizes from about 2 to about 4 microns and are predominantly oligolamellar, that is, containing more than one or at least a few lipid bilayer shells. The REVs may be sized by extrusion, if desired, to give oligolamellar vesicles having e.g. a maximum selected size between about 0.05 and about 1.5 μm.

Preparations of large multilamellar vesicles (LMLV) or REV can be further treated, e.g., by way of extrusion, sonication or high pressure homogenization, to produce small unilamellar vesicles (SUV's), which are characterized by sizes in the range of about 0.03 micron to about 0.1 micron. Alternatively, SUV's can be formed directly by homogenization of an aqueous dispersion of lipids.

Other methods for adding additional components to liposomal compositions include methods wherein an aqueous liposome dispersion is co-lyophilized with other components and the resulting solid redispersed to form MLV. Another method (A. Adler, Cancer Biother. 10: 293, 1995) provides addition of an aqueous solution of the agent to be encapsulated to a t-butanol solution of lipids. The mixture is sonicated and lyophilized, and the resulting powder is rehydrated.

In a preferred embodiment, the liposomes the present invention containing a high dose of immunogen of are prepared by rehydration of a lyophilized lipid complement with water or an aqueous ethanol solution, the immunogen being contained in the lipid complement or in the aqueous ethanol solution. In particular embodiments the aqueous ethanol solution is from about 1% to about 10% ethanol by volume. Preferably the aqueous ethanol solution is about 5% ethanol by volume.

Liposome compositions containing an entrapped agent can again be treated after final sizing, if necessary, to remove the free (non-entrapped) agent. Conventional separation techniques, such as centrifugation, diafiltration, and ultrafiltration are suitable for this purpose. The composition can also be sterilized by filtration through a conventional 0.45 micron filter. In order to form the compositions of the current invention, the concentration of immunogen conjugate in the liposomes can be chosen to give a protein/lipid molar ratio from about 1:100 to about 1:1000, at 100% encapsulation, after filtration.

The liposome preparations of the invention have been found stable over the long term. Upon storage at 4° C., the liposome carrier was still fully stable after 1 year, such that the entrapped immunogenic agents retained 75-95% of their initial activity for at least 3-6 months, with IL-2 liposomes being particularly stable. The IL-2 and antigen loaded liposomes showed less than 10% loss of activity for up to 6 months.

Stabilizers may also be added to the liposomal compositions. For example, when a metal chelator, such as Desferal™ or diethylenetriamine pentaacetic acid (DTPA) was included in the lyophilization medium at a concentration of 100 μM, the IL-2 biological activity loss was reduced even further. For more extended storage, the compositions may be converted to a dry lyophilized powder, which is stable for much more than a year, and can be hydrated to form an aqueous suspension as needed before use.

In humans, an effective antigen dose may be in the range of about 50 μg to about 5 mg.

Parenteral administration can be by injection, which is e.g., intraperitoneal (i.p.), subcutaneous (s.c.), intravenous (i.v.), intramuscular (i.m.) or transdermal . The vaccine can also be administered across mucosal membranes, such as intranasally, rectally, vaginally, or perorally.

Multilamellar vesicles of the invention have been found capable of encapsulating large amounts of hydrophilic proteins for vaccine formulations containing, e.g., an anti-gastrin conjugate, G17DT, or an anti-GnRH conjugate, GnRHDT (also designated D17DT). One procedure of such an encapsulation is described in the Example 1 although the method is not limited to the particular liposomal immunogens of the examples.

The conjugates were prepared according to methods disclosed in the co-assigned U.S. Pat. Nos. 5,023,077 and 5,468,494 (G17DT), and 5,688,506 (GnRHDT) and 6,132,720, which entire methods have been incorporated herein by reference. The sequence analogs of these conjugates have been described above.

Moreover, CCK-2/gastrin receptor immunogen (disclosure incorporated herein by reference to co-assigned pending application Ser. No. 09/076,372), and hCG immunogen as described above are suitable substances for encapsulation in the afore-described liposomes. The use of examples of human gastrin analogs or fragments is not meant to exclude gastrin hormones of other animal species in the practice of this invention.

The liposomes of this invention can be utilized to prepare specific treatments for a broad spectrum of pathologic conditions, including vaccines and drug delivery systems against cancer, infectious disease and other disorders. The antigens targeted by liposome based vaccines can be soluble molecules or cell-associated molecules. Specific examples, provided to illustrate the breadth of application of this invention without limiting the scope of invention, include:

For treatment of cancers derived from the gastrointestinal tract and stimulated to grow by the hormone gastrin-17 and/or by glycine extended gastrin-17, such as pancreatic or gastric cancer, a liposome based vaccine according to the present invention containing the immunogen G17DT can be used to induce neutralizing antibodies against gastrin-17.

For reproductive tract tumors stimulated to grow by gonadal steroids, including but not limited to such reproductive tract tumors as prostatic carcinoma, a liposome based vaccine according to the present invention containing the immunogen D17DT can be used to induce neutralizing antibodies against GnRH. These induced antibodies lead to the elimination of gonadal steroid synthesis and prevent further hormone-stimulated growth of the prostatic carcinoma.

For infectious disease caused by Streptococcus pneurnoniae, liposomes according to the present invention can be formulated with streptococcal coat carbohydrate antigens conjugated to DT, to induce neutralizing antibodies against pneumococcus.

For influenza, liposomes according to the present invention containing inactivated influenza virus of one or more serotypes can be employed to induce immunity to these viruses.

For tetanus, liposomes according to the present invention can be formulated with tetanus toxoid, to induce neutralizing antibodies against tetanus toxin.

For gastroesophageal reflux disease caused by stomach acid reflux into the esophagus, liposomes according to the present invention containing a G17DT conjugate can be formulated to induce antibodies that neutralize serum gastrin and thereby reduce stomach acid content.

### EXAMPLE 1

### Liposomal Encapsulation

The bilayer forming components which can be used for the production of multilamellar liposomes (MLV) include dimyristoyl phosphatidylcholine (DMPC) and dimyristoyl phosphatidylglycerol (DMPG) (Lipoid, Genzyme or Avanti Polar Lipids).

MLV were prepared by freeze-drying overnight mixtures of G17DT or GnRHDT immunogen with or without nor-MDP adjuvant in aqueous solution and tert-butanol solution of lipids (either neutral DMPC alone or a 9:1 by weight ratio mixture of DMPC:DMPG dissolved in tert-butanol). To prepare lyophilized liposomes as a suspension for injection, the method of hydration and suspension has major effects on the protein encapsulation by the liposomes. Best results are obtained when hydration is achieved by adding the water in small increments.

In assessing the effect of the ratio of lipid/protein (w/w) on protein encapsulation, it was found that increasing the amount of lipid to attain a DMPC/protein ratio of 1000:1 did not result in a more advantageous protein encapsulation than with the ratio of 500:1. Therefore, most of the working embodiments of the invention focused on lipid-to-protein or DMPC/protein ratio of 500:1. (See Table I and Example 6). The invention also provides liposomes having a lipid/protein ratio of 300:1 which has been found to be optimal.

The efficiency of encapsulation of GnRHDT and G17DT (hereafter also identified as “protein”) by liposomes was calculated after centrifugation by quantification of the amount of protein in liposome pellet fraction and the free non-encapsulated proteins present in the aqueous supernatant phase. The protein was quantified using a modified Lowry method. Peterson G. L. 1983. “Determination of total protein.” Methods Enzymol. 91: 95-119. In order to assess the level of contamination of the aqueous phase by liposomes, the amount of phospholipid was determined by quantifying organic phosphate using the modified Bartlett method: Bartlett, G. R. 1959; and “Phosphorus assay in column chromatography” J. Biol. Chem. 234 : 446-468; and Y. Barenholz et al. “Liposome preparation and related techniques” 1993, In Lysosome Technology Vol. 1, 2^{nd} ed. (Gregoriadis, G. Ed.) CRC Press, Boca Raton, Fla., pp. 526-616.

TABLE I
Effect of lipid/protein weight ratio on % protein encapsulation.

| | Lipid/Protein | Protein encapsulation |
|---|---|---|
| Liposome formulation | ratio (w/w) | (%) |
| DMPC/G17DT | 500:1 | 89.4 ± 7.86 |
| DMPC/G17DT | 300:1 | 90 |
| DMPC/DMPG/G17DT | 500:1 | 86.0 ± 2.5 |
| DMPC/GnRHDT | 500:1 | 97.05 ± 2.5 |
| DMPC/GnRHDT | 300:1 | 86 |

The efficacy of protein encapsulation of the negatively charged DMPC/DMPG liposome formulation composed of 90% DMPC and 10% DMPG at lipid/protein ratio 500:1, was about the same as liposomes formulated with 100% DMPC.

The hydration and suspension of the lyophilized samples was achieved by adding purified water using the Waterpro Ps HPLC/Ultrafilter Hybrid, which provides low levels of total organic carbon and inorganic ions in sterile pyrogen-free water (pH 5.4). The pH of the solution was determined on the day of hydration. Although the actual pH of the various test preparation may have ranged from about 5.2 to 6.7, it was of no discernible consequence to the efficacy of the preparation. The liposomal formulations were kept at a lipid/protein weight ratio of 500:1, such as DMPC with protein; DMPC with protein and adjuvant; DMPC/DMPG with protein; and the DMPC/DMPG mixture with protein and adjuvant.

The particle size distribution of liposome dispersions was determined at 25° C. by dynamic light scattering (DLS) with Coulter model N4 SD as described by Y. Barenholz, et al. (ibid.) or by a Coulter counter (Coulter Multisizer Accucomp). Contaminant or unloaded liposomes were in the range of 0.2-0.8 μm.

The liposome size distribution ranging of about 1.3 to 1.8 μm was confirmed by dynamic light scattering (DLS) 80 to 100% of the particles.

The sizes of the resultant liposomes measured by the Coulter counter consistently confirming average volumes varying from 3.7 to 5, (SD of ±3).

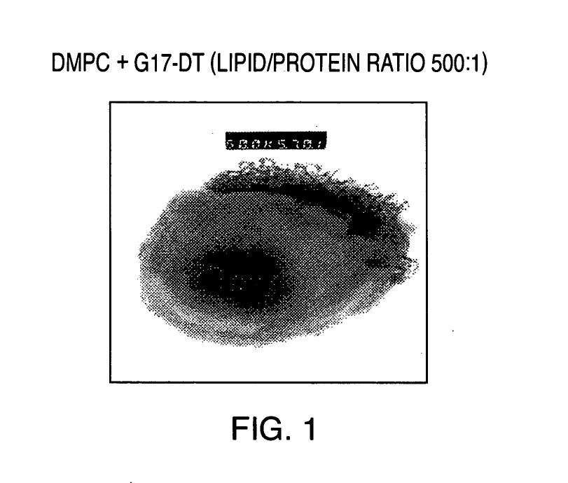

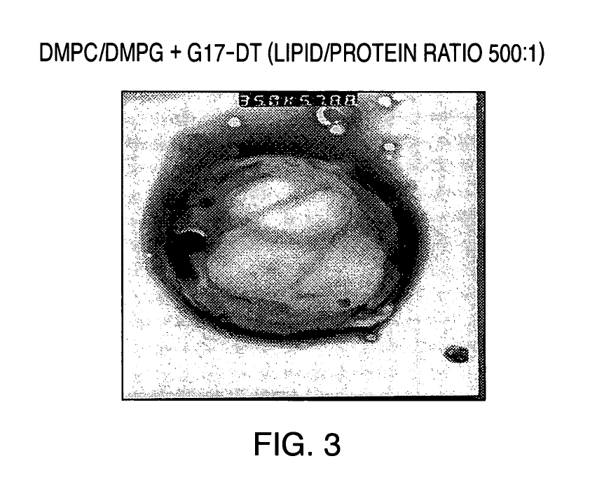

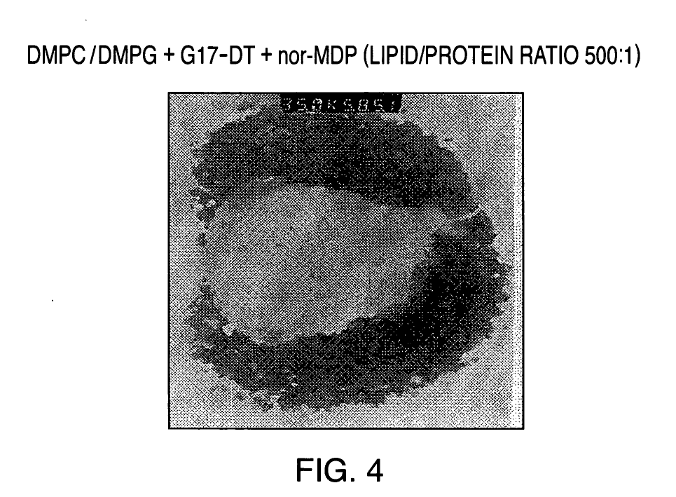

Samples of liposomes containing GnRHDT (i.e. D17DT) were visualized by electron microscopy and measured using negative staining. FIG. 1-FIG. 4 are electron micrographs depicting the two different liposomal vaccines negatively stained in phosphotungstate sodium (Lipid/protein weight ratio of 500:1). The particle diameters obtained from a number of electron micrographs showed on average about 50 particles measuring a mean of about 1-2.5 μm for each of the liposome formulations.

The experiments also established the efficacy of the high lipid to protein ratio liposome preparation method to entrap hydrophilic protein content, as e.g., the above-identified conjugates. In particular, the instant multilamellar vesicles were found to hold high concentrations or quantities of conjugates of DT, or other water-soluble proteins, in part located on the lipid bilayer membranes and in part completely internalized within the membrane enclosure or shell.

The following examples show the effect increased vaccine dosage on tissue reactogenicity.

### EXAMPLE 2

### Lower Dosage G17DT Liposome Compared to G17DT Emulsion (w/o)

G17DT conjugate was encapsulated in an aqueous liposomal suspension at conjugate dosages of 100 μg or 200 μg protein. This liposomal G17DT vaccine preparation was tested in female rabbits (in groups of three) by injections on days 0, 28, and 56, respectively, and compared to the prior art 100 μg dose of the G17DT emulsion control.

Sera samples were collected at 14 days intervals over the course of the 84 day study, and tested for anti-gastrin antibody titers by ELISA. The liposome preparations were found at 100 μg dose/0.2 ml volume to have induced a peak mean response of 10,370 titer on day 70, after 3 injections. All other liposome samples showed titers of 5,000 or less, indicating that it at least three injections were required to induce titers over 10,000 and that these titers were not sustained for an extended time. Doubling the administered dose to 200 μg/0.4 ml resulted in a mean titer of 11,162 in sera collected 14 days after injection 3. The increased dose was somewhat more effective, since a mean titer of 9,553 (or ˜10,000) was attained 14 days after injection 2, indicating a measurable improvement over the 100 μg dose regimen. However, these responses were of short duration, as the mean titers of sera collected on the other bleed days (day 0-14-28-56-84) were all lower than 5,000. Although an improvement was achieved by doubling the conjugate dose delivered by the liposome formulation, the responses were of short duration. Therefore these liposomes were not considered practical as vaccines for clinical use, where as the same regimen using an emulsion dose of 100 μg G17DT in ISA 703 (Group 13) produced an average rabbit serum titer of anti-gastrin antibody in excess of 10,000 from day 42 onward.

Apparently, this outcome with liposomal immunogen is significantly less effective than the results set forth below (Example 3).

However, the liposome formulations were very well tolerated at the injection site, producing no visible tissue reaction. As this was an improvement over the water-in-oil emulsion immunization, the apparent protective effect of liposome encapsulation of the antigen was tested at a higher antigen loads. As confirmed by the further examples described below, administration of relatively large amounts of water-soluble immunogens (1-3 mg/dose) achieved clinically effective immune responses without significant tissue reaction.

### EXAMPLE 3

### G17DT-Liposome

As shown in foregoing Example 2, doses of conjugate that are normally quite effective when administered in Montanide® ISA 703 (“ISA 703”) modified emulsions, are not sufficiently effective when encapsulated in liposomes. However, administering an order of magnitude larger doses of liposome-encapsulated G17DT (distributed over a large number of particles) increased efficacy. Despite the dosage size, only very low tissue reactogenicity could be visualized, as described below. In addition, the immunomodulatory effect of the cytokine, IL-2, in liposome preparation, administered as a separate supplemental injection, was found to distinctly enhance the antibody response.

Thus, the present example was useful to evaluate the immunogenicity and local tolerance values of high doses of hG17DT (either 1.5 or 3.0 mg) formulated in the aforedescribed liposomes when administered with and without IL-2 (i.e. doses of 0, 1,000, 10,000, or 100,000 cu IL-2 in liposomes) in a series of separate supplemental injections. The efficacy of the formulations was compared with aqueous buffer formulations (PBS) containing G17DT (1.5 or 3.0 mg doses), as well as a Montanide® ISA 703 emulsion containing G17DT conjugate (100 μg dose in a 0.2 ml emulsion volume), as controls.

Specifically, thirteen rabbit groups (n=4 per group) were immunized with the G17DT immunogens and IL-2 supplements encapsulated in liposomes. The liposomes were injected intramuscularly (i.m.) with 1.0 ml dose volumes (Groups 1-11) or subcutaneously (s.c.) with 2.0 ml dose volumes (Group 12). The animals of Group 1 received 100 μg G17DT in ISA 703 for injection 1, then 1.5 mg G17DT in liposomes (no IL-2) for injections 2 and 3. The ISA 703 emulsions were injected i.m. with 0.2 ml dose volumes in Groups 1 and 13. Each rabbit was injected i.m. with in 0.1 ml dose volumes of the IL-2 formulations (all groups except 1, 10, 11, and 13). The injections were administered in a series of three sets of injections, given on days 0, 28 and 56. Serum samples were collected at 14-day intervals over the 84 days of treatment at which time all rabbits were euthanized and scored for injection site reactions. Biopsies from two animals per group were evaluated by microscopic examination.

Anti-G17 antibody responses were measured by ELISA, a direct binding assay method, wherein antibody binding to wells coated with gastrin target antigen was detected indirectly by using an anti-antibody-enzyme complex plus enzyme substrate.

### EXPERIMENTAL PROCEDURE

G17DT Immunogen Formulations

The test materials consisted of various formulations of G17DT Immunogen and IL-2, which were prepared from the following components.

1. hG17DT; hG17 (1-9) pGlu-Gly-Pro-Trp-Leu-Glu-Glu-Glu-Glu-Ser-Ser-Pro-Pro-Pro-Pro-Cys coupled to an immunogenic carrier. (SEQ ID NO: 18 in the Sequence Listing);

2. Phosphate Buffered Saline (PBS): [0.017M Na₂HPO₄+0.001M KH₂PO₄+0.14M NaCl, pH 7.2];

3. Montanide®ISA 703: (Seppic; Paris, France);

4. DMPC: hG17DT Liposomes;

5. DMPC/DMPG: Liposomes for cytokines;

6. IL-2: 3×10⁶ cu stock solution; and

7. Sterile Saline: 0.9% NaCl in distilled water, filtered through 0.2 μm syringe filter.

The hG17DT immunogen was prepared in accordance with methods disclosed in U.S. Pat. No. 5,468,494, which methods have been incorporated herein by reference.

Test Formulations

The G17DT Immunogens and IL-2 supplements were aseptically formulated in the combinations shown in Table A. For all liposome and IL-2 formulations, the appropriate volume of sterile saline was added into each vial in 100 μl increments with vigorous vortexing between additions. The ISA 703 emulsion was prepared using a standard hand-mixing method using a 70:30 (oil:aqueous phase, wt:wt) ratio. PBS was used as diluent to prepare the aqueous phase. The test materials were dispensed into syringes and stored under refrigeration (2-8° C.).

In Vivo Protocol:

Adult, virgin female, pathogen-free New Zealand white rabbits were used in the study. The rabbits were grouped (n=4) and immunized with the G17DT immunogens as shown in Table B. Three sets of injections per rabbit, on days 0, 28, and 56, in dose volumes as shown. Intramuscular (i.m.) or subcutaneous injections (s.c.) were given in the hind legs following a standard protocol, with the first injection set given in the right leg, the second injection set given in the left leg, and the third injection set given in the right leg higher than the first set of injections. The injection sites were tattooed for later identification.

To assess immunogenicity, sera were prepared from blood samples obtained from each rabbit every 14 days until day 84, when the rabbits were euthanized. Blood (15 ml per bleed) was collected from marginal ear veins using an 18 gauge needle, then stored at 2-8° C. overnight to allow for clot shrinkage. The samples were then centrifuged (400×g) and the sera were removed by pipette and frozen as individual samples at −10° to −25° C. until assayed.

Antibody Assay:

Anti-Gastrin antibody titers were measured in the sera samples by ELISA. Sera tested for antibodies were collected on test days 0, 14, 28, 42, 56, 70, and 84.

Gross Pathology:

All the test animals were examined for gross injection site pathology, on day 84. Injection sites were located by tattoos, the skin was refected to fully expose the muscle, and a transverse incision was made completely through the muscle at each injection site. Tissues were visually evaluated for gross pathology on a scale of 0-3, where a score of 0 indicated that the tissue appeared normal, and a score of 3 indicated the presence of an extensive inflammatory reaction throughout the injection area of the tissue. Scores of 1 and 2 represent intermediate levels of local reaction.

Microscopic Pathology Observations

After grading for gross pathology, two rabbits per treatment group were randomly selected for microscopic pathology observation. The i.m. injection sites were biopsied by excising a 2 to 2.5 cm length of quadriceps muscle with a scalpel and immediately submerging the tissue specimens in a minimum volume of 25 ml of buffered formalin. Each sample was placed in a separate vial and allowed to fix in the formalin for a minimum of 24 hours. The vials were processed for histopathological evaluation of a region of the biopsy for microscopic examination, after paraffin embedding, sectioning at 5 μm thickness, mounting, and H and E staining. Individual histology scores and the scoring system of Example 3 are given in Table C.

Statistical Analysis:

Both the mean and median anti-Gastrin titers were calculated (Table C) from the individual antibody titer and group responses for selected bleeds were compared using the Student's t-Test. The results of the statistical analyses comprising mean titers of group B (G17DT emulsion) are given in Table D.

Mean injection site reaction scores were calculated from the gross pathology observations. Mean histology scores were calculated and are given in Table D.

Immunologic Results:

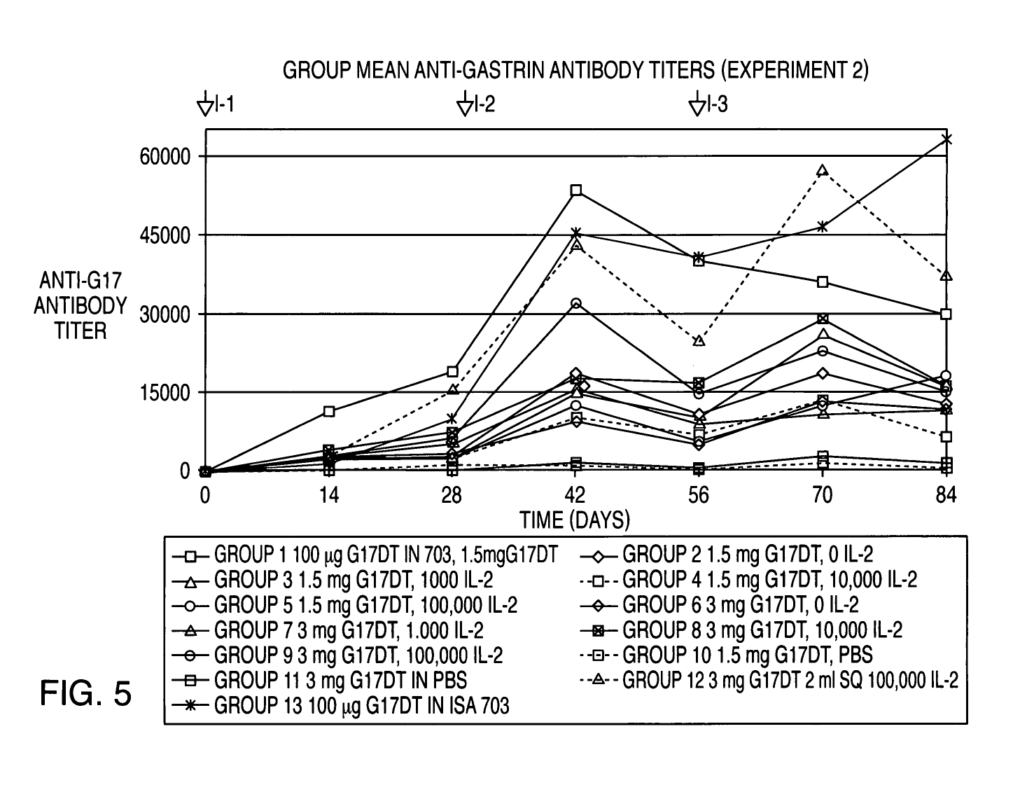

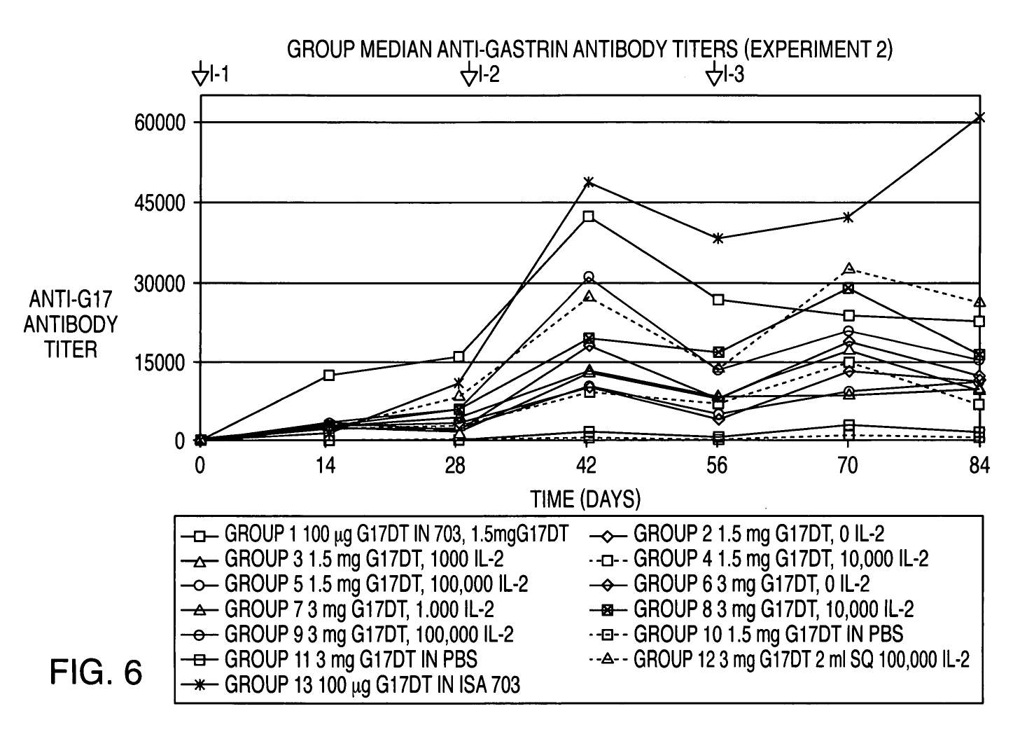

The anti-hG17 antibody responses generated by each group over the course of the 84 day immunogenicity test in vivo were measured by ELISA. Mean antibody titers are given in Table E. The mean titers are plotted in FIG. 5, with the median titer plots shown in FIG. 6.

As shown in the drawings (FIG. 5 and FIG. 6), the control G17DT immunogen emulsion, formulated in Montanide® ISA 703 and delivering 100 μg G17DT/dose (Group 13), induced responses characterized by the high peak anti-hG17 antibody titer (day 84) and the strongest sustained antibody production throughout the study. Titers in excess of 10,000 were reached by day 42 and maintained thereafter. The responses of rabbits injected i.m. with liposome preparations were lower in titer and tended to present shorter, more highly defined booster responses after injections #2 and #3. However, several liposome formulations, administered to test rabbit groups 2, 3, 7, 9, and 12, induced and sustained titers in excess of 10,000. The Group 1 peak titers subsequent to the third injection were not statistically significantly higher than those of the liposome i.m. injection groups (Groups 2-8), with the exception of Groups 4 and 6. The responses of Group 4 (1.5 mg G17DT, 10,000 cu IL-2) and Group 6 (3.0 mg G17DT, 0 IL-2) were characterized by comparatively low standard deviations at the peak mean titer, thus accounting for the statistical significance in comparison with the Group 1 controls.

There was no significant difference between the peak mean responses of Group 2 (1.5 mg, no IL-2) and Group 6 (3.0 mg, no IL-2), indicating that the dose increase from 1.5 to 3.0 mg G17DT did not measurably enhance immunogenicity. The remaining two test groups, including Group 1 (injection #1 emulsion, with subsequent injections #2 and #3 being 1.5 mg G17DT liposomal preparations) and Group 12 (s.c.-injected liposomes) elicited responses which had a titer about equal to the Group 13 control, though the response kinetics more closely resembled the i.m. liposome groups. There was no measurable boost in titers following injection #3 in Group 12; although the mean antibody levels were relatively stable from day 56 (injection #3) to the end of the study, suggesting that the third dose may have sustained antibody production. As expected, Groups 9 and 10 were low responders to solutions with the high antigen doses, 1.5 mg and 3.0 mg G17DT, in PBS, respectively.

IL-2 did not significantly affect antibody levels at the 1.5 mg G17DT dose level, as shown in Table D. At the 3.0 mg dose, only Group 8 (10,000 cu IL-2) differed significantly from Group 6 (no IL-2); however, at this dose of conjugate the groups (8 and 9) receiving the two higher doses of IL-2 had elevated antibody titers compared to the low IL-2 dose (Group 7) and no IL-2 (Group 6) groups. These data suggest that the immunogenicity of the 3.0 mg G17DT dose was enhanced by the supplemental administration of 10,000 to 100,000 cu of stimulatory IL-2.

The injection site reaction grades were assessed visually in all rabbits on day 84. As the data show, injection site reactions were minimal for all groups except Group 12 (subcutaneous) and Group 13 (standard emulsion preparation). These two subject groups presented scores >1 in 2 of 4 animals in Group 12, and in 1 of 4 animals in Group 13 at the third immunogen injection site. In Groups 1-11, minor reaction scores of 0.5 were observed in 14 out of 96 sites (15%) of IL-2 administration. Sites injected with immunogen in these groups for the most part (66%) received scores of 0.5 (87 out of 132 sites), with 33% scored at 0 (43 out of 132 sites). Thus, visual assessment indicated that the liposome preparations were very well tolerated when administered i.m.

Microscopic Pathology Observations

The mean histopathology results on day 84 are shown in Table D. Microscopic pathology readings of the injection site biopsies were generally in accord with the gross visual evaluation results, with the highest scores occurring either at sites that received immunogen formulated in ISA 703, or where the immunogen was administered subcutaneously.

Inflammatory reactions were minimal for nearly all of the liposome i.m. injection sites. The sites injected with immunogen tended to have slightly higher scores than IL-2 sites. Of the liposome injection sites exhibiting inflammation, several were noted to contain moderate to pronounced calcification (6 sites) and/or significant scarring of muscle fibers (4 sites, 3 of which were also calcified). The muscle reaction scores seen in Group 13 are typical for water-in-oil emulsions. However, the score of 2.5 at site 1 in Rabbit #124 of Group 1, is somewhat unusual for a primary injection site graded 84 days after dosing. Higher scores were noted in Group 12, where liposomes were given subcutaneously. It should be noted that the visual and histologic reaction grading systems are independent and not correlated against one another. Generally, the histology reaction scores exceed the visual scores. Nevertheless, significantly less muscle inflammation was induced by the liposomes than the water-in-oil emulsions.

Conclusion:

The results of the experiment of Example 3 demonstrate that liposomes formulated at a high lipid-to-protein ratio (500:1) delivering 1.5 and 3.0 mg G17DT in a large number of MLV, induces anti-hG17 antibody levels following i.m. administration roughly 25% of those elicited by the potent formulation comprising Montanide ISA 703 emulsion yet high enough to be clinically effective. Simultaneously, very low tissue reactogenicity was observed despite the significant increased amount of vaccine. The immunogenicity of the 1.5 and 3.0 mg dose formulations was equivalent. Immunogenicity of the 3.0 mg dose was enhanced by supplemental administration of IL-2 mixed with liposomes, at doses of 10,000 and 100,000 cu IL-2. IL-2 had no effect on immunogenicity of the 1.5 mg dose. Subcutaneous (s.c.) administration of the 3.0 mg immunogen dose significantly enhanced immunogenicity, as did the priming with the initial Montanide emulsion formulation in Group 1 followed by boosts with the 1.5 mg liposomes. The reactogenicity of the liposome formulations of high immunogen content was significantly decreased after i.m. administration, but not by s.c. administration. These results indicate that high protein liposomal preparations of G 17DT compare favorably with about one tenth the amount of immunogen formulated as a Montanide ISA 703 emulsion, by significantly reducing reactogenicity, while providing effective immunogenicity.

TABLE A
IMMUNOGEN FORMULATIONS (Example 3)

| Immunogen | | Conjugate (mg) | Dose |
|---|---|---|---|
| Lot No. | Vehicle | or IL-2 (cu) | Volume |
| 1A | DMPC/DMPG liposome | 1.5 mg | 1 ml |
| 1B | DMPC/DMPG liposome | 3.0 mg | 1 ml |
| 1C | DMPC/DMPG liposome | 3.0 mg | 2 ml |
| 1D | PBS solution | 1.5 mg | 1 ml |
| 1E | PBS solution | 3.0 mg | 1 ml |
| 1F | DMPC/DMPG liposome | 0 cu | 0.1 ml |
| 1G | DMPC/DMPG liposome | 1,000 cu | 0.1 ml |
| 1H | DMPC/DMPG liposome | 10,000 cu | 0.1 ml |
| 1I | DMPC/DMPG liposome | 100,000 cu | 0.1 ml |
| 1J | Montanide ISA 703 | 100 μg | 0.2 ml |
| | Emulsion | | |

TABLE B
RABBIT DOSAGE GROUPS (Example 3)

| | Rabbits/ | hG17DT Dose | Injection 1 | Injection 1′ | Injection 2 | Injection 2′ | Injection 3 | Injection 3′ |
|---|---|---|---|---|---|---|---|---|
| Group # | Group (n) | (IL-2 Dose) | (Day 0) | (Day 0) | (Day 28) | (Day 28) | (Day 56) | (Day 56) |
| 1 | 4 | 100 μg/1.5 mg | 1J | Na | 1A | na | 1A | na |
| | | emulsion | 0.2 ml i.m. | | 1 vial | | 1 vial | |
| | | (na/0 cu) MLV | | | | | | |
| 2 | 4 | 1.5 mg | 1A | 1F | 1A | 1F | 1A | 1F |
| | | (0 cu) MLV | 1 vial i.m. | 0.1 ml | 1 vial | 0.1 ml | 1 vial | 0.1 ml |
| 3 | 4 | 1.5 mg | 1A | 1G | 1A | 1G | 1A | 1G |
| | | (1,000 cu) MLV | 1 vial i.m. | 0.1 ml | 1 vial | 0.1 ml | 1 vial | 0.1 ml |
| 4 | 4 | 1.5 mg | 1A | 1H | 1A | 1H | 1A | 1H |
| | | (10,000 cu) MLV | 1 vial i.m. | 0.1 ml | 1 vial | 0.1 ml | 1 vial | 0.1 ml |
| 5 | 4 | 1.5 mg | 1A | 1I | 1A | 1I | 1A | 1I |
| | | (100,000 cu) MLV | 1 vial i.m. | 0.1 ml | 1 vial | 0.1 ml | 1 vial | 0.1 ml |
| 6 | 4 | 3.0 mg (1 ml) | 1B | 1F | 1B | 1F | 1B | 1F |
| | | (0 cu) MLV | 1 vial i.m. | 0.1 ml | 1 vial | 0.1 ml | 1 vial | 0.1 ml |
| 7 | 4 | 3.0 mg (1 ml) | −1B | 1G | 1B | 1G | 1B | 1G |
| | | (1,000 cu) MLV | 1 vial i.m. | 0.1 ml | 1 vial | 0.1 ml | 1 vial | 0.1 ml |
| 8 | 4 | 3.0 mg (1 ml) | 1B | 1H | 1B | 1H | 1B | H |
| | | (10,000 cu) MLV | 1 vial i.m. | 0.1 ml | 1 vial | 0.1 ml | 1 vial | 0.1 ml |
| 9 | 4 | 3.0 mg (1 ml) | 1B | 1I | 1B | 1I | 1B | 1I |
| | | (100,000 cu) MLV | 1 vial | 0.1 ml | 1 vial | 0.1 ml | 1 vial | 0.1 ml |
| 10 | 4 | 1.5 mg (PBS) | 1D | Na | 1D | na | 1D | na |
| | | solution | 1 ml | | 1 ml | | 1 ml | |
| 11 | 4 | 3.0 mg (PBS) | 1E | Na | 1E | na | 1E | na |
| | | solution | 1 ml | | 1 ml | | 1 ml | |
| 12 | 4 | 3.0 mg (2 ml) | 1C | 1I | 1C | 1I | 1C | 1I |
| | | (100,000 cu) s.c. | 2 vials | 0.1 ml | 2 vials | 0.1 ml | 2 vials | 0.1 ml |
| 13 | 4 | 100 μg (ISA 703) | J | Na | 1J | na | 1J | na |
| | | emulsion | 0.2 ml | | 0.2 ml | | 0.2 ml | |

′= separate injection of IL-2
1 vial = 1 ml
MLV = liposome

TABLE C
(Example 3)
INJECTION SITE REACTIONS ON DAY 84

| | | | Immunogen | IL-2 | Immunogen | IL-2 | Immunogen | IL-2 |
|---|---|---|---|---|---|---|---|---|
| Group # | | | Site 1 | Site 2 | Site 3 | Site 4 | Site 5 | Site 6 |
| 1. | 100 μg G17DT in 703 | Mean | 0.4 | n/a | 0.4 | n/a | 0.1 | n/a |
| | emulsion and 1.5 mg | No. >1 | 0 | n/a | 0 | n/a | 0 | n/a |
| | G17DT in MLV; i.m. | | | | | | | |
| 2. | 1.5 mg G17DT | Mean | 0.4 | 0.1 | 0.5 | 0.1 | 0.4 | 0.0 |
| | 0 cu IL-2; | No. >1 | 0 | 0 | 0 | 0 | 0 | 0 |
| | in MLV; i.m. | | | | | | | |
| 3. | 1.5 mg G17DT | Mean | 0.4 | 0.1 | 0.5 | 0.3 | 0.5 | 0.0 |
| | 1000 cu IL-2; | No. >1 | 0 | 0 | 0 | 0 | 0 | 0 |
| | in MLV; i.m. | | | | | | | |
| 4. | 1.5 mg G17DT 10,000 | Mean | 0.3 | 0.1 | 0.5 | 0.1 | 0.6 | 0.0 |
| | cu IL-2; in MLV; i.m. | No. >1 | 0 | 0 | 0 | 0 | 0 | 0 |
| 5. | 1.5 mg G17DT | Mean | 0.4 | 0.0 | 0.5 | 0.0 | 0.5 | 0.0 |
| | 100,000 cu IL-2; | No. >1 | 0 | 0 | 0 | 0 | 0 | 0 |
| | in MLV; i.m. | | | | | | | |
| 6. | 3 mg G17DT 0 cu IL- | Mean | 0.4 | 0.0 | 0.5 | 0.0 | 0.5 | 0.0 |
| | in MLV; i.m. | No. >1 | 0 | 0 | 0 | 0 | 0 | 0 |
| 7. | 3 mg G17DT 1000 cu | Mean | 0.5 | 0.3 | 0.5 | 0.3 | 0.4 | 0.0 |
| | IL-2; in MLV; i.m. | No. >1 | 0 | 0 | 0 | 0 | 0 | 0 |
| 8. | 3 mg G17DT | Mean | 0.3 | 0.1 | 0.5 | 0.3 | 0.5 | 0.0 |
| | 10,000 cu IL-2; | No. >1 | 0 | 0 | 0 | 0 | 0 | 0 |
| | in MLV; i.m. | | | | | | | |
| 9. | 3 mg G17DT 100,000 | Mean | 0.3 | 0.0 | 0.4 | 0.0 | 0.5 | 0.0 |
| | cu IL-2; in MLV; i.m. | No. >1 | 0 | 0 | 0 | 0 | 0 | 0 |
| 10. | 1.5 mg G17DT | Mean | 0.0 | n/a | 0.0 | n/a | 0.0 | n/a |
| | in PBS; in | No. >1 | 0 | n/a | 0 | n/a | 0 | n/a |
| | MLV; i.m. | | | | | | | |
| 11. | 3 mg G17DT in PBS; | Mean | 0.0 | n/a | 0.0 | n/a | 0.0 | n/a |
| | in MLV; i.m. | No. >1 | 0 | n/a | 0 | n/a | 0 | n/a |
| 12. | 3 mg G17DT 2 ml | Mean | 0.1 | 0.1 | 0.6 | 0.0 | 1.3 | 0.0 |
| | 100,000 cu IL-2; in | No. >1 | 0 | 0 | 0 | 0 | 2 | 0 |
| | MLV; s.c. | | | | | | | |
| 13. | 100 μg G17DT in | Mean | 0.5 | n/a | 0.6 | n/a | 1.1 | n/a |
| | ISA 703 emulsion. | No. >1 | 0 | n/a | 0 | n/a | 1 | n/a |

TABLE D
(Example 3)
MEAN INJECTION SITE HISTOLOGY SCORES ON DAY 84

| | | Immunogen | IL-2 | Immunogen | IL-2 | Immunogen | IL-2 |
|---|---|---|---|---|---|---|---|
| Group # | | Site 1 | Site 2 | Site 3 | Site 4 | Site 5 | Site 6 |
| 1. | 100 μg G17DT in 703 | 1.5 | n/a | 0.5 | n/a | 0.8 | n/a |
| | emulsion | | | | | | |
| | 1.5 mg G17DT | | | | | | |
| | in MLV; i.m. | | | | | | |
| 2. | 1.5 mg G17DT | 0.5 | 0.3 | 0.8 | 0.3 | 1.3 | 0.0 |
| | 0 cu IL-2; in MLV; i.m. | | | | | | |
| 3. | 1.5 mg G17DT | 0.5 | 0.5 | 1.0 | 0.8 | 0.5 | 0.3 |
| | 1000 cu IL-2; | | | | | | |
| | in MLV; i.m. | | | | | | |
| 4. | 1.5 mg G17DT | 0.3 | 0.5 | 0.8 | 0.5 | 1.0 | 0.3 |
| | 10,000 cu IL-2; | | | | | | |
| | in MLV; i.m. | | | | | | |
| 5. | 1.5 mg G17DT | 0.5 | 0.5 | 0.8 | 0.5 | 0.5 | 0.0 |
| | 100,00 cu IL-2; | | | | | | |
| | in MLV; i.m. | | | | | | |
| 6. | 3 mg G17DT | 0.5 | 0.5 | 1.0 | 0.3 | 1.3 | 0.3 |
| | 0 cu IL-2; in MLV; i.m. | | | | | | |
| 7. | 3 mg G17DT | 0.5 | 0.5 | 1.3 | 0.5 | 0.5 | 0.0 |
| | 1000 cu IL-2; | | | | | | |
| | in MLV; i.m. | | | | | | |
| 8. | 3 mg G17DT; | 0.5 | 0.5 | 1.0 | 0.8 | 0.8 | 0.5 |
| | 10,000 cu IL-2; | | | | | | |
| | in MLV; i.m. | | | | | | |
| 9. | 3 mg G17DT | 0.3 | 0.5 | 1.0 | 0.3 | 1.0 | 0.5 |
| | 100,000 cu IL-2; | | | | | | |
| | in LV; i.m. | | | | | | |
| 10. | 1.5 mg G17DT in PBS | 0.3 | n/a | 0.3 | n/a | 0.0 | n/a |
| | i.m. | | | | | | |
| 11. | 3 mg G17DT in PBS | 0.3 | n/a | 0.3 | n/a | 0.0 | n/a |
| | i.m. | | | | | | |
| 12. | 3 mg G17DT 2 ml MLV | 0.5 | 0.5 | 1.5 | 1.8 | 2.0 | 0.5 |
| | 100,000 cu IL-2; s.c. | | | | | | |
| 13. | 100 μg G17DT in | 0.8 | n/a | 2.0 | n/a | 2.3 | n/a |
| | ISA 703 | | | | | | |
| | emulsion i.m. | | | | | | |

**Contains moderate to marked calcification
## significant scarring of muscle fibers identified.
Histopathology Scoring
0-0.5: No inflammation or other histopathological abnormality.
1.0-1.5: Mild active or residual chronic inflammation.
2.0-2.5: Moderate active or chronic inflammation.
3.0: Severe chronic or active inflammation

TABLE E
(Example 3)
RABBIT SERUM ANTI-GASTRIN ANTIBODY RESPONSES

| | | | Day | Day | Day | Day | Day | Day |
|---|---|---|---|---|---|---|---|---|
| Group # | | Day 0 | 14 | 28 | 42 | 56 | 70 | 84 |
| Group 1 | Mean | 0 | 11,616 | 19,000 | 53,450 | 40,164 | 36,075 | 30,025 |
| 100 μg G17DT in 703, 1.5 mg | Median | 0 | 12,201 | 15,700 | 42,100 | 26,650 | 23,700 | 22,600 |
| G17DT in MLV, i.m. | S.D. | POOL | 9,023 | 8,955 | 38,495 | 39,218 | 31,628 | 21,819 |
| Group 2 | Mean | 0 | 2,816 | 2,530 | 18,800 | 10,934 | 18,700 | 12,702 |
| 1.5 mg G17DT, 0 cu IL-2 | Median | 0 | 2,769 | 2,168 | 17,850 | 7,799 | 18,650 | 11,950 |
| in MLV, i.m. | S.D. | POOL | 1,693 | 1,790 | 4,001 | 7,284 | 3,966 | 5,416 |
| Group 3 | Mean | 0 | 1,840 | 3,175 | 15,134 | 10,848 | 26,325 | 15,801 |
| 1.5 mg G17DT, 1000 cu IL-2 | Median | 0 | 1,934 | 1,837 | 12,950 | 8,162 | 17,300 | 9,542 |
| in MLV, i.m. | S.D. | POOL | 438 | 3,365 | 7,483 | 8,307 | 22,747 | 16,063 |
| Group 4 | Mean | 0 | 2,479 | 2,227 | 10,177 | 6,951 | 13,514 | 6,323 |
| 1.5 mg G17DT, 10,000 cu | Median | 0 | 2,804 | 2,529 | 8,988 | 6,962 | 14,550 | 6,594 |
| IL-2 in MLV, i.m. | S.D. | POOL | 1,102 | 1,086 | 4,540 | 4,534 | 4,457 | 2,058 |
| Group 5 | Mean | 0 | 1,956 | 2,724 | 12,465 | 5,525 | 12,429 | 18,297 |
| 1.5 mg G17DT, 100,000 cu | Median | 0 | 1,980 | 2,339 | 10,375 | 4,971 | 8,957 | 11,151 |
| IL-2 in MLV, i.m. | S.D. | POOL | 684 | 2,086 | 8,093 | 2,623 | 8,850 | 17,814 |
| Group 6 | Mean | 0 | 2,713 | 3,440 | 9,818 | 5,445 | 12,975 | 11,561 |
| 3 mg G17DT, 0 cu IL- | Median | 0 | 2,953 | 2,965 | 10,250 | 4,222 | 13,050 | 11,150 |
| 2 in MLV, i.m. | S.D. | POOL | 904 | 1,286 | 1,201 | 2,721 | 866 | 3,043 |
| Group 7 | Mean | 0 | 2,497 | 5,573 | 15,732 | 9,200 | 11,203 | 11,714 |
| 3 mg G17DT, 1,000 cu IL- | Median | 0 | 2,336 | 4,167 | 13,254 | 8,219 | 8,800 | 9,870 |
| 2 in MLV, i.m. | S.D. | POOL | 1,003 | 3,854 | 11,722 | 6,140 | 7,646 | 7,931 |
| Group 8 | Mean | 0 | 4,221 | 7,414 | 17,550 | 16,850 | 28,825 | 16,425 |
| 3 mg G17DT, 10,000 cu IL-2 | Median | 0 | 3,048 | 5,946 | 19,050 | 16,800 | 28,650 | 16,250 |
| in MLV, i.m. | S.D. | POOL | 2,863 | 4,601 | 5,231 | 4,279 | 4,863 | 3,154 |
| Group 9 | Mean | 0 | 3,990 | 6,519 | 32,054 | 14,838 | 23,098 | 14,981 |
| 3 mg G17DT, 100,000 cu | Median | 0 | 3,100 | 5,716 | 30,700 | 13,511 | 20,900 | 15,100 |
| IL-2 in MLV, i.m. | S.D. | POOL | 2,827 | 4,410 | 20,123 | 8,801 | 15,296 | 7,098 |
| Group 10 | Mean | 0 | 39 | 1,342 | 918 | 432 | 1,646 | 533 |
| 1.5 mg G17DT in PBS, i.m. | Median | 0 | 18 | 74 | 337 | 177 | 993 | 443 |
| | S.D. | POOL | 56 | 2,586 | 1,261 | 557 | 1,660 | 302 |
| Group 11 | Mean | 0 | 122 | 127 | 1,537 | 754 | 2,776 | 1,462 |
| 3 mg G17DT in PBS, i.m. | Median | 0 | 121 | 116 | 1,559 | 518 | 2,806 | 1,392 |
| | S.D. | POOL | 105 | 140 | 1,427 | 852 | 1,995 | 1,130 |
| Group 12 | Mean | 0 | 3,237 | 15,518 | 43,150 | 25,025 | 57,225 | 37,325 |
| 3 mg G17DT 2 ml S.C., | Median | 0 | 3,178 | 8,578 | 27,150 | 14,150 | 32,550 | 26,450 |
| 100,000 cu IL-2 in MLV. | S.D. | POOL | 1,850 | 17,797 | 32,990 | 24,874 | 59,367 | 29,297 |
| Group 13 | Mean | 0 | 1,574 | 9,860 | 45,269 | 41,025 | 46,450 | 63,175 |
| 100 μg G17DT in ISA 703 | Median | 0 | 1,495 | 10,913 | 48,550 | 38,150 | 42,300 | 60,650 |
| Emulsion | S.D. | POOL | 752 | 5,900 | 31,551 | 25,306 | 37,223 | 43,159 |

### EXAMPLE 4

### Lower Dosage GnRH Compared to GnRH without Emulsion

Initial experiments compared reactogenicity and immunogenicity of liposomal GnRHDT vaccine and the water in oil emulsion GnRH vaccine. GnRHDT conjugate (i.e. D17DT) was encapsulated in an aqueous liposome suspension with conjugate dosages of 100 μg to 1000 μg protein. The liposomal GnRH vaccine was tested in female rabbits with an i.m. injection on day 0, 14 and 42, respectively, and compared to the GnRHDT emulsion vaccine of about the same dosage.

Sera were collected from the rabbits every 14 days from day 0 through day 70, and tested for anti-GnRH antibodies titers by ELISA. It was found that the i.m. injections of liposomes delivering 100 μg dose/0.2 ml volume induced a mean peak titer of 2,004 on day 70 after three injections. All other serum samples showed mean peak response titers of 582, indicating that at least three injections would be required to induce a titer of 2,000. Moreover, the antibody titers were not sustained, but significantly declined shortly after peak titers were attained.

Doubling the immunogen conjugate dose to 200 μg/0.4 ml liposomes resulted in a mean titer of 2,060 in sera collected 14 days after the third injection on day 56, remaining at a mean titer of 2,005 on day 70. The increased dosage was found already more effective by inducing mean titer of 768 when assessed 14 days after injection #2, as compared to the low titer of only 166 induced by the dose of 100 μg/0.2 ml. Further increases of the dose, such as 500 μg/1.0 ml and 1000 μg/1.0 ml antigen raised the mean titers to 2,962 and 3,494, respectively, on day 56, declining to 2,133 and 2,889, respectively, on day 70. Thus, these responses were of short duration, with the antibody titers responsive to the liposome immunization falling off significantly from day 28 to day 42 and day 56 to day 70. However, it appeared that the increased conjugate doses led to increases in anti-GnRH antibody responses.

While the increased liposome dosage of 1 mg/ml GnRHDT conjugates showed the desired low reactogenicity, the immune response still fell short of the required threshold of efficacy in eliciting a titer of over 5000 found sufficient to neutralize GnRH activity immunological sterilization.

### EXAMPLE 5

### GnRHDT (i.e. D17DT)

As described below, an experiment was conducted to assess the effect upon immunogenicity and reactogenicity when incorporating relatively high doses of GnRHDT in the form of D17DT into liposomes. The study also investigated the immunomodulatory effect of administering IL-2 with liposomes as a separate supplemental injection. Previous studies as described in Example 4, had demonstrated that liposomal vaccine preparation would overcome the problem of increased reactogenicity found in animals immunized with increased emulsion dosages (Example 4).

Emulsions with dosages of 100 μg and 200 μg GnRHDT in Montanide ISA 703 had been sufficient in most instances for clinically effective immunization, while generally causing relatively moderate tissue reactions. However, occasionally the need arose requiring dosages as high 500 μg or 1000 μg in 0.2 to 0.5 ml injection volumes of emulsion. These increased dosages were discovered to increase the occurrence of more severe tissue reaction of the treated patient. Therefore, in view of the 200 μg per 0.2 ml dosage limit regarding reactogenicity, other more ameliorating means of immunization was required.

It was found that using a high ratio of lipids to protein the liposomes could encapsulate a large amount of immunogen by distributing the water-soluble protein in a large number of small vesicles. The present experiment evaluated large doses (either 1.5 or 3.0 mg) of GnRHDT (i.e. D17DT) formulated in high lipid ratio liposomes when administered with and without IL-2 (0, 1,000, 10,000, or 100,000 cu doses) as a separate supplemental injection. These formulations were prepared by methods described in Example 1 and compared to aqueous formulations containing GnRHDT in PBS (1.5 or 3.0 mg conjugate in 0.2 mL dose volumes), as well as Montanide® ISA 703 emulsion containing GnRHDT (100 μg in a 0.2 ml dose volume) (summarized in Table 1). Thirteen groups of 4 rabbits each were immunized with the GnRHDT immunogen and IL-2 supplements (see Table 2). Liposomes were injected intramuscularly (i.m.) with 1.0 mL dose volumes in Groups 1-9 and subcutaneously (s.c.) with 2.0 mL dose volumes in Group 12. Group 1 received 100 μg GnRHDT in ISA 703 for injection 1, followed with 1.5 mg GnRHDT in MLV liposomes (no IL-2) for injections #2 and #3. The ISA 703 emulsions were injected i.m. in 0.2 mL dose volumes in control Groups 1 and 13. Groups 2-9 and 12 were injected i.m. with the IL-2 formulations in 0.1 mL dose volumes on the same study days that they received the immunogen. Groups 10 and 11 were injected with 1.5 and 3.0 mg GnRHDT conjugate in aqueous PBS solutions respectively. The injections were administered on days 0, 28 and 56. Serum samples were collected at 14-day intervals over 84 days and scored visually for injection site reactions, biopsies from two animals per group were evaluated by microscopic examination. Anti-GnRH antibody responses were measured by ELISA (Table 3).

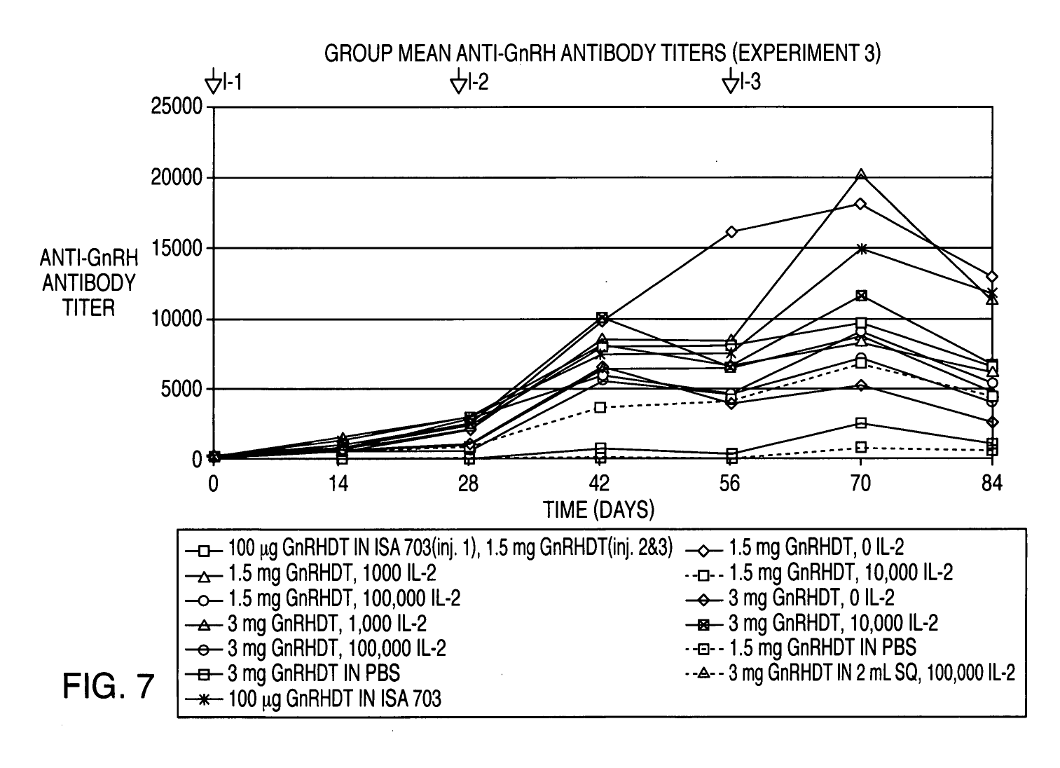

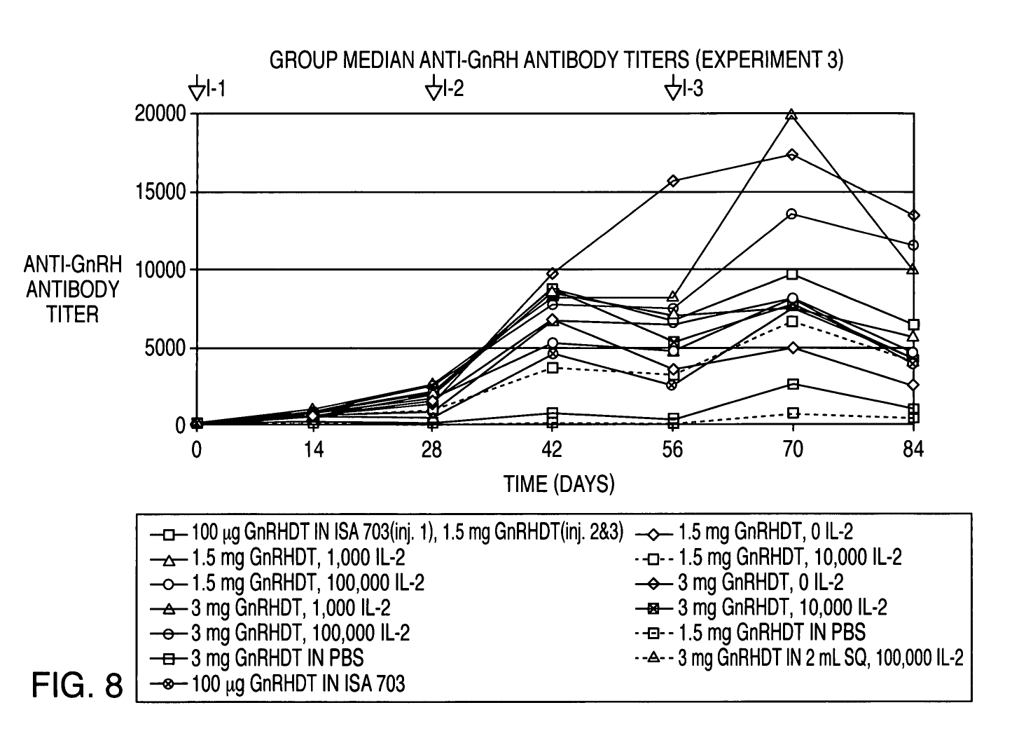

The experiment of this Example shows that liposomes formulated at high lipid to protein ratio as vehicles to deliver 1.5 and 3.0 mg GnRHDT can induce anti-GnRH antibody responses following i.m. or s.c. (3.0 mg only) administration in rabbits (See FIGS. 7 and 8). Assays of the dose response showed that 3.0 mg of conjugate is more immunogenic than 1.5 mg. Moreover, the 3.0 mg dose, not supplemented with IL-2, induced even higher anti-GnRH antibody titers than the Montanide ISA 703 immunogen emulsion control. Surprisingly, immunogenicity of the liposome vaccines was not enhanced by supplemental injection of IL-2; in fact, at the 3.0 mg dose, IL-2 may have even reduced the response.

When compared to the liposome preparations given i.m., the s.c. administration of the 3.0 mg dose in Group 12 enhanced immunogenicity significantly, whereas priming the rabbit with the Montanide formulation (Group 1) followed by boosts with the 1.5 mg liposomes (GnRH in MLV) only showed slightly improved titers. The local muscle tissue reactogenicity of the injected liposome formulations was substantially subdued in comparison to the Montanide ISA 703 immunogen emulsion controls. The antibody responses were similar to the emulsion controls, including groups injected with 3.0 mg i.m. without added IL-2 and with 3.0 mg s.c., while the histology scores were consistently lower, and visual scores were improved considerably. In contrast, treatment with the high protein solutions of GnRHDT in PBS did not cause strong tissue reactions in muscle while the titer of anti-GnRH antibodies was ineffectively low.

The results demonstrate that the multilamellar liposomal preparations of GnRHDT, formulated to contain an order of magnitude higher doses, compare favorably with Montanide ISA 703 GnRHDT immunogen emulsion in terms of both immunogenicity and reactogenicity.

### EXPERIMENTAL PROCEDURE

GnRHDT Immunogen Formulations:

The test materials consisted of various formulations of GnRHDT Immunogen and IL-2, which were prepared from the following components.

1. GnRHDT: GnRH (1-10) Ser-1-DT also designated D17DT;

2. Phosphate Buffered Saline (PBS): [0.017M Na₂HPO₄+0.001M KH₂PO₄+0.14M NaCl, pH 7.2];

3. Montanide® ISA 703 (Seppic; Paris, France);

4. DMPC: GnRHDT Liposomes;

5. DMPC/DMPG Liposomes for IL-2 or other cytokines.

6. IL-2: 3×10⁶ cu; and

7. Sterile Saline: 0.9% NaCl in distilled water, filtered through 0.2 μm syringe filter.

Test Formulations

The GnRHDT immunogens and IL-2 supplements were formulated under aseptic conditions in the various combinations shown in Table 1. To suspend the liposomes, the appropriate volume of sterile saline was injected into each vial in 100 μL increments with vigorous vortexing between small additions. The modifying agent IL-2 was dissolved in sterile saline, and then mixed with the DMPC/DMPG liposomes to give the appropriate concentration of IL-2. The ISA 703 emulsion was prepared using a standard hand-mixing method using a 70:30 (oil: aqueous phase, wt:wt) ratio. PBS was used as diluent to prepare the aqueous phase. The test materials were dispensed into syringes and stored under refrigeration (2-8° C.) before use.

In vivo protocol:

Fifty-two adult, virgin female, specific pathogen-free, New Zealand white rabbits were used in the study. The rabbits were grouped (n=4) and immunized with the GnRHDT-immunogens. Three sets of injections were given per rabbit, on days 0, 28, and 56, in dose volumes as shown in Table 2. Intramuscular or subcutaneous injections were given in the hind legs following a standard protocol, with the first injection set given in the right leg, the second injection set given in the left leg, and the third injection set given in the right leg higher than the first set of injections. The injection sites were tattooed for later identification.

To assess immunogenicity, sera were prepared from blood samples obtained from each rabbit every 14 days until day 84. Blood (15 mL per bleed) was collected from marginal ear veins using an 18 gauge needle, then stored at 2-8° C. overnight to allow for blood clot shrinkage. The samples were then centrifuged (400×g) and the sera were removed by pipette and frozen as individual samples at −10 to −25° C. until assayed.

Antibody assay:

Anti-GnRH antibody titers were measured in the sera samples by ELISA. Sera tested for antibodies were collected on test days 0, 14, 28, 42, 56, 70, and 84. (Table 3)

Gross Pathology:

Gross injection site pathology was assessed in all rabbits on day 84, as described in Example 3.

Microscopic Pathology:

After grading for gross pathology, two rabbits per group were randomly selected for microscopic pathology observation. The i.m. injection sites were biopsied by excising a 2 to 2.5 cm length of quadriceps muscle with a scalpel and immediately submerging the tissue specimens in a minimum volume of 25 mL of Histochoice™. Each sample was placed in a separate vial and allowed to fix in the solution for a minimum of 24 hours prior to histopathological evaluation.

Results:

Statistical Analysis

Mean and median anti-GnRH titers were calculated for each group (Table 3) and responses for selected bleeds were compared using the Student t-Test. Mean injection site reaction scores on day 84 were calculated from the gross pathology observations and are given in Table 4. Mean histology scores were calculated and are given in Table 5.

Immunologic Results:

The anti-GnRH antibody responses generated by each group over the course of the 84-day in vivo test were measured by ELISA. Median and mean antibody titers are given in Table 3. The mean titers are plotted in FIG. 7, and median titers in FIG. 8.

As shown in the FIGS. 7 and 8, the control GnRHDT immunogen formulated in Montanide® ISA 703 and delivered at a dose of 100 μg GnRHDT/dose (Group 13) induced high anti-GnRH antibody titers that peaked on Day 70. The responses of rabbits treated with injections with liposome preparations injected i.m. (Groups 2-9) were generally lower in titer than those induced by the emulsion control; however, were of sufficient titer to be clinically effective in the reduction or neutralization of GnRH of the immunized animal. An exception to this general result was Group 6 (3.0 mg GnRHDT, no IL-2), wherein the mean/median titers exceeded those in control Group 13. The responses of all groups were appropriately boosted upon each injection. In fact, statistical comparison of the mean peak titers following the third injection indicated that the responses induced by i.m. injection of the liposomes at either dose of conjugate were not significantly below those of the Montanide/immunogen emulsion control (Group 13).

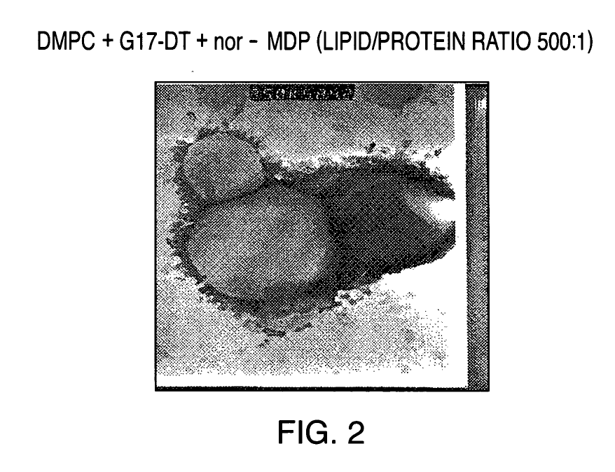

In general, liposomes delivering the 3.0 mg dose of GnRHDT were more immunogenic than those with the 1.5 mg dose (FIG. 2). This is particularly relevant when considering the responses in relation to the requisite titer to neutralize the biological activity of GnRH thereby mediating infertility or suppressing gonadal steroid synthesis. Previous Aphton studies have indicated that a titer of 5,000 is efficacious in rabbits sterilization. As depicted in FIG. 2, rabbits immunized i.m. (Groups 2-9) with the 3.0 mg GnRHDT dose appeared to induce effective titers faster and sustain them longer than those immunized with the 1.5 mg dose. This difference was statistically significant. From this perspective, the 3.0 mg GnRHDT dose response was superior to the 1.5 mg dose. The two remaining test Groups, 1 and 12, produced anti-GnRH responses that exceeded those of all other liposome groups, except Group 6. Group 12 (3 mg GnRHDT, s.c.) was the highest responding group in the study, suggesting that the subcutaneous injection route might be more conducive to the induction of high antibody titers by liposome formulations. As expected, injections of Groups 9 and 10 with 1.5 mg and 3.0 mg G17DT in PBS solution, respectively, reduced only low responders.

The cytocine IL-2 did not affect antibody levels significantly in combination with the 1.5 mg GnRHDT dose. At the 3.0 mg dose, Group 6 without IL-2, produced titers that were significantly higher than the other 3.0 mg liposome preparations injected i.m. in Groups 7, 8, and 9. Moreover, there were no significant differences between the responses of Groups 7-9. These data suggest that liposome delivered immunogenicity in rabbits was not enhanced by supplementation with IL-2.

As the data presented in Table 4 shows, injection site reactions were minimal for all groups, scoring no higher than 1.0. While all Group 13 animals (control emulsion preparation) presented scores of 1.0 at the third immunogen injection site, only one rabbit in each of the liposome groups 1, 7, 9 and 12 had a score of 1.0 namely, at the site of the third injection. The majority (74%) of i.m. liposome injection sites received scores of 0.5 and 23% received scores of 0. In addition, the immunological adjuvant IL-2 was very well tolerated, with 88% receiving scores of 0. Thus, visual assessment indicated that the i.m. liposome preparations were very well tolerated.

Microscopy Pathology Observations (Table 5)

Microscopic pathology readings of the injection site biopsies were generally in accord with the gross evaluation results, with the highest scores occurring at sites that received immunogen formulated in ISA 703. The muscle reaction scores seen in Group 13 are in accord with those normally observed with Montanide® ISA 703 formulations. Scores slightly lower than those induced by the emulsion were obtained where the immunogen was administered s.c. (Group 12) and in rabbits that produced a significant response to i.m. injection (Group 6). Inflammatory reactions were minimal for nearly all of the other liposome i.m. injection sites. The sites injected with immunogen tended to have slightly higher scores than IL-2 sites, the latter generally exhibiting very little evidence of inflammation with the exceptions of Groups 6 and 9, both at site 1. It should be noted that the visual and histologic reaction grading systems are independent and not correlated against one another. Generally, the numerical histology reaction scores exceed the visual scores. In sum, the evaluations established that the liposomes appear to induce significantly less muscle inflammation than do the water-in-oil emulsions, despite increased injection volumes.

Conclusion:

The results of the Example 5 demonstrate that liposomes formulated at a lipid:protein ratio about 500:1 by weight to deliver 1.5 and 3.0 mg GnRHDT distributed over large number of relatively small lipoid particles, induce anti-GnRH antibody responses following i.m. or s.c. (3.0 mg) administration in rabbits. A dose response was evident, with 3.0 mg of conjugate eliciting more immunogenicity than 1.5 mg. It was surprising that the 3.0 mg dose, not supplemented with IL-2, induced higher anti-GnRH titers than the Montanide ISA 703 control. Thus, immunogenicity was not enhanced by supplemental injection of IL-2; in fact, at the 3.0 mg dose, IL-2 may have effected a reduction of the response. In comparison with the high lipid protein ratio liposome preparations regardless of whether they were given either by i.m. or s.c., administration of the 3.0 mg dose significantly enhanced immunogenicity, whereas priming with the Montanide formulation followed by boosts with the 1.5 mg liposomes improved titers only slightly. Despite the increased volume of the vaccine dose, the reactogenicity of the liposome formulations was significantly decreased in comparison with the much lower amounts of the GnRHDT: Montanide ISA 703 emulsion controls. Thus, the histology scores of the injection loci were lower, and their visual scores were considerably improved over the emulsion controls although antibody responses were comparable to the emulsion controls. These results demonstrate that liposomal preparations of doses of GnRHDT as large as 3.0 mg, formulated at high lipid to protein ratios compare favorably with immunogen prepared as a Montanide ISA 703 emulsion in that reactogenicity is significantly reduced or even eliminated, while effective anti-GnRH antibody titers are produced. Moreover, the study demonstrates that potentially toxic effects of cytokine stimulation of the patient's immune system can be avoided by the present liposome vaccine by omitting the cytokines or similar agent entirely from the composition or treatment.

TABLE 1
IMMUNOGEN FORMULATIONS (Example 5)

| | | Conjugate or IL-2 | Dose |
|---|---|---|---|
| Immunogen | Vehicle | Content | Volume |
| 2A | DMPC/DMPG(MLV) | 1.5 mg GnRHDT | 1 mL |
| 2B | DMPC/DMPG(MLV) | 3.0 mg GnRHDT | 1 mL |
| 2C | DMPC/DMPG(MLV) | 3.0 mg GnRHDT | 2 mL |
| 2D | PBS solution | 1.5 mg GnRHDT | 1 mL |
| 2E | PBS solution | 3.0 mg GnRHDT | 1 mL |
| 2F | DMPC/DMPG(MLV) | 0 cu IL-2 | 0.1 mL |
| 2G | DMPC/DMPG(MLV) | 1,000 cu IL-2 | 0.1 mL |
| 2H | DMPC/DMPG(MLV) | 10,000 cu IL-2 | 0.1 mL |
| 2I | DMPC/DMPG(MLV) | 100,000 cu IL-2 | 0.1 mL |
| 2J | Montanide ISA 703 | 100 μg GnRHDT | 0.2 mL |
| | emulsion | | |

TABLE 2
RABBIT DOSAGE GROUPS (Example 5)

| | | GnRHDT | | | | | | |
|---|---|---|---|---|---|---|---|---|
| | Rabbits/ | Dose | Injection 1 | Injection 1′ | Injection 2 | Injection 2′ | Injection 3 | Injection 3′ |
| Group # | Group | (IL-2 Dose) | (Day 0) | (Day 0) | (Day 28) | (Day 28) | (Day 56) | (Day 56) |
| 1 | 4 | 100 μg/1.5 mg | 2J | NA | 2A | NA | 2A | NA |
| | | (na/0 cu) MLV | 0.2 mL | | 1 vial | | 1 vial | |
| 2 | 4 | 1.5 mg | 2A | 2F | 2A | 2F | 2A | 2F |
| | | (0 cu) MLV | 1 vial | 0.1 mL | 1 vial | 0.1 mL | 1 vial | 0.1 mL |
| 3 | 4 | 1.5 mg | 2A | 2G | 2A | 2G | 2A | 2G |
| | | (1,000 cu) MLV | 1 vial | 0.1 mL | 1 vial | 0.1 mL | 1 vial | 0.1 mL |
| 4 | 4 | 1.5 mg | 2A | 2H | 2A | 2H | 2A | 2H |
| | | (10,000 cu) MLV | 1 vial | 0.1 mL | 1 vial | 0.1 mL | 1 vial | 0.1 mL |
| 5 | 4 | 1.5 mg | 2A | 2I | 2A | 2I | 2A | 2I |
| | | (100,000 cu) MLV | 1 vial | 0.1 mL | 1 vial | 0.1 mL | 1 vial | 0.1 mL |
| 6 | 4 | 3.0 mg (1 mL) | 2B | 2F | 2B | 2F | 2B | 2F |
| | | (0 cu) MLV | 1 vial | 0.1 mL | 1 vial | 0.1 mL | 1 vial | 0.1 mL |
| 7 | 4 | 3.0 mg (1 mL) | 2B | 2G | 2B | 2G | 2B | 2G |
| | | (1,000 cu) MLV | 1 vial | 0.1 mL | 1 vial | 0.1 mL | 1 vial | 0.1 mL |
| 8 | 4 | 3.0 mg (1 mL) | 2B | 2H | 2B | 2H | 2B | 2H |
| | | (10,000 cu) MLV | 1 vial | 0.1 mL | 1 vial | 0.1 mL | 1 vial | 0.1 mL |
| 9 | 4 | 3.0 mg (1 mL) | 2B | 2I | 2B | 2I | 2B | 2I |
| | | (100,000 cu) MLV | 1 vial | 0.1 mL | 1 vial | 0.1 mL | 1 vial | 0.1 mL |
| 10 | 4 | 1.5 mg (PBS) | 2D | NA | 2D | NA | 2D | NA |
| | | solution | 1 mL | | 1 mL | | 1 mL | |
| 11 | 4 | 3.0 mg (PBS) | 2E | NA | 2E | NA | 2E | NA |
| | | solution | 1 mL | | 1 mL | | 1 mL | |
| 12 | 4 | 3.0 mg (2 mL) | 2C | 2I | 2C | 2I | 2C | 2I |
| | | (100,000) MLV | 2 vials | 0.1 mL | 2 vials | 0.1 mL | 2 vials | 0.1 mL |
| 13 | 4 | 100 mg (ISA 703) | 2J | NA | 2J | NA | 2J | NA |
| | | emulsion | 0.2 mL | | 0.2 mL | | 0.2 mL | |

1 vial = 1 ml
′= separate injetion

TABLE 3
RABBIT SERUM ANTI-GnRH ANTIBODY RESPONSES (Example 5)

| | | | Day | Day | Day | Day | Day | Day |
|---|---|---|---|---|---|---|---|---|
| Group # | | Day 0 | 14 | 28 | 42 | 56 | 70 | 84 |
| Group 1 | Mean | 0 | 577 | 2,255 | 10,115 | 6,735 | 11,651 | 6,643 |
| 100 μg GnRHDT in | Median | 0 | 547 | 1,866 | 8,405 | 5,216 | 8,062 | 4,269 |
| ISA703 (inj. 1) emulsion | S.D. | Pool | 126 | 1,406 | 7,016 | 4,037 | 8,126 | 5,116 |
| 1.5 mg GnRHDT | | | | | | | | |
| MLV (inj. 2&3) i.m. | | | | | | | | |
| Group 2 | Mean | 0 | 702 | 1,211 | 6,682 | 4,001 | 5,305 | 2,600 |
| 1.5 mg GnRHDT, 0 cu IL-2 | Median | 0 | 524 | 1,046 | 6,549 | 3,590 | 4,936 | 2,555 |
| in MLV i.m. | S.D. | Pool | 504 | 660 | 586 | 1,388 | 3,289 | 1,788 |
| Group 3 | Mean | 0 | 803 | 1,232 | 6,430 | 6,569 | 8,835 | 4,835 |
| 1.5 mg GnRHDT, 1,000 cu IL-2 | Median | 0 | 823 | 995 | 6,820 | 6,311 | 8,135 | 4,132 |
| in MLV i.m. | S.D. | Pool | 382 | 950 | 3,263 | 3,630 | 2,083 | 2,075 |
| Group 4 | Mean | 0 | 899 | 940 | 3,674 | 4,184 | 6,753 | 4,346 |
| 1.5 mg GnRHDT, 10,000 cu IL-2 | Median | 0 | 677 | 790 | 3,599 | 3,229 | 6,570 | 4,055 |
| in MLV i.m. | S.D. | Pool | 926 | 790 | 2,686 | 3,910 | 4,058 | 2,049 |
| Group 5 | Mean | 0 | 672 | 717 | 5,715 | 4,637 | 7,300 | 4,126 |
| 1.5 mg GnRHDT, 100,000 cu IL-2 | Median | 0 | 395 | 308 | 4,415 | 2,546 | 7,694 | 3,814 |
| in MLV i.m. | S.D. | Pool | 647 | 897 | 3,963 | 5,018 | 1,704 | 1,160 |
| Group 6 | Mean | 0 | 777 | 2,047 | 9,949 | 16,375 | 18,350 | 13,065 |
| 3 mg GnRHDT, 0 cu IL-2 | Median | 0 | 650 | 1,297 | 9,651 | 15,600 | 17,300 | 13,450 |
| in MLV i.m. | S.D. | Pool | 502 | 1,911 | 4,404 | 5,110 | 5,149 | 3,739 |
| Group 7 | Mean | 0 | 1,645 | 3,156 | 8,219 | 6,729 | 8,486 | 6,233 |
| 3 mg GnRHDT, 1,000 cu IL-2 | Median | 0 | 1,015 | 2,463 | 8,453 | 7,092 | 7,641 | 5,643 |
| in MLV i.m. | S.D. | Pool | 1,668 | 2,610 | 2,001 | 2,136 | 2,171 | 2,494 |
| Group 8 | Mean | 0 | 936 | 2,481 | 8,110 | 8,158 | 9,742 | 6,502 |
| 3 mg GnRHDT, 10,000 cu | Median | 0 | 810 | 1,923 | 8,637 | 6,814 | 9,596 | 6,410 |
| IL-2 in MLV i.m. | S.D. | Pool | 572 | 1,732 | 1,901 | 3,855 | 2,909 | 1,819 |
| Group 9 | Mean | 0 | 907 | 3,077 | 5,953 | 4,820 | 9,156 | 5,388 |
| 3 mg GnRHDT, 100,000 cu | Median | 0 | 750 | 1,512 | 5,209 | 4,799 | 8,169 | 4,684 |
| IL-2 in MLV i.m. | S.D. | Pool | 405 | 3,610 | 4,020 | 2,045 | 2,754 | 2,430 |
| Group 10 | Mean | 0 | 0 | 0 | 154 | 97 | 811 | 369 |
| 1.5 mg GnRHDT in PBS | Median | 0 | 0 | 0 | 129 | 90 | 767 | 358 |
| i.m. | S.D. | Pool | 1 | 1 | 61 | 29 | 225 | 145 |
| Group 11 | Mean | 0 | 16 | 28 | 807 | 407 | 2,582 | 967 |
| 3 mg GnRHDT in PBS | Median | 0 | 14 | 8 | 641 | 287 | 2,646 | 988 |
| i.m. | S.D. | Pool | 14 | 43 | 496 | 289 | 958 | 444 |
| Group 12 | Mean | 0 | 748 | 2,240 | 8,714 | 8,615 | 20,450 | 11,449 |
| 3 mg GnRHDT in | Median | 0 | 541 | 2,435 | 8,047 | 8,234 | 19,900 | 10,038 |
| 2 mL 100,000 cu IL-2 | S.D. | Pool | 445 | 715 | 3,342 | 2,981 | 5,510 | 3,660 |
| MLV; s.c. | | | | | | | | |
| Group 13 | Mean | 0 | 1,494 | 3,070 | 7,612 | 7,688 | 15,166 | 11,869 |
| 100 μg GnRHDT in ISA 703 | Median | 0 | 951 | 2,602 | 7,744 | 7,402 | 13,600 | 11,550 |
| emulsion | S.D. | Pool | 1,643 | 2,173 | 2,492 | 2,984 | 9,040 | 5,302 |

TABLE 4
MEAN INJECTION SITE REACTIONS ON DAY 84 (Example 5)

| | | | Immunogen | IL-2 | Immunogen | IL-2 | Immunogen | IL-2 |
|---|---|---|---|---|---|---|---|---|
| Group # | | | Site 1 | Site 2 | Site 3 | Site 4 | Site 5 | Site 6 |
| 1. | 100 μg GnRHDTin ISA703 | Mean | 0.5 | N/A | 0.3 | N/A | 0.5 | N/A |
| | (inj. 1) emulsion 1.5 mg | No. >1 | 0 | N/A | 0 | N/A | 0 | N/A |
| | GnRHDT(inj. 2&3) | | | | | | | |
| | MLV i.m. | | | | | | | |
| 2. | 1.5 mg GnRHDT, 0 cu IL-2 | Mean | 0.3 | 0.0 | 0.5 | 0.0 | 0.3 | 0.0 |
| | in MLV; i.m. | No. >1 | 0 | 0 | 0 | 0 | 0 | 0 |
| 3. | 1.5 mg GnRHDT, 1,000 cu | Mean | 0.4 | 0.0 | 0.1 | 0.0 | 0.5 | 0.0 |
| | IL-2 in MLV; i.m. | No. >1 | 0 | 0 | 0 | 0 | 0 | 0 |
| 4. | 1.5 mg GnRHDT, | Mean | 0.3 | 0.0 | 0.4 | 0.0 | 0.5 | 0.1 |
| | 10,000 cu IL-2 | No. >1 | 0 | 0 | 0 | 0 | 0 | 0 |
| | in MLV; i.m. | | | | | | | |
| 5. | 1.5 mg GnRHDT, 100,000 | Mean | 0.0 | 0.0 | 0.5 | 0.1 | 0.5 | 0.1 |
| | cu IL-2 in MLV; i.m. | No. >1 | 0 | 0 | 0 | 0 | 0 | 0 |
| 6. | 3 mg GnRHDT, 0 cu IL-2 | Mean | 0.3 | 0.3 | 0.5 | 0.0 | 0.5 | 0.0 |
| | in MLV; i.m. | No. >1 | 0 | 0 | 0 | 0 | 0 | 0 |
| 7. | 3 mg GnRHDIT, 1,000 cu | Mean | 0.4 | 0.1 | 0.5 | 0.1 | 0.6 | 0.0 |
| | IL-2 in MLV; i.m. | No. >1 | 0 | 0 | 0 | 0 | 0 | 0 |
| 8. | 3 mg GnRHDT, 10,000 | Mean | 0.4 | 0.1 | 0.5 | 0.3 | 0.4 | 0.0 |
| | cu IL-2 MLV; i.m. | No. >1 | 0 | 0 | 0 | 0 | 0 | 0 |
| 9. | 3 mg GnRHDT, 100,000 | Mean | 0.4 | 0.3 | 0.5 | 0.1 | 0.6 | 0.0 |
| | cu IL-2 MLV | No. >1 | 0 | 0 | 0 | 0 | 0 | 0 |
| 10. | 1.5 mg GnRHDT, | Mean | 0.0 | N/A | 0.0 | N/A | 0.0 | N/A |
| | in PBS solution i.m. | No. >1 | 0 | N/A | 0 | N/A | 0 | N/A |
| 11. | 3 mg GnRHDT, | Mean | 0.0 | N/A | 0.0 | N/A | 0.0 | N/A |
| | in PBS solution i.m. | No. >1 | 0 | N/A | 0 | N/A | 0 | N/A |
| 12. | 3 mg GnRHDT, in 2 mL | Mean | 0.1 | 0.4 | 0.4 | 0.3 | 0.6 | 0.3 |
| | 100,000 cu IL-2 MLV; s.c. | No. >1 | 0 | 0 | 0 | 0 | 0 | 0 |
| 13. | 100 μg GnRHDT in ISA | Mean | 0.4 | N/A | 0.6 | N/A | 1.0 | N/A |
| | 703 emulsion; i.m. | No. >1 | 0 | N/A | 0 | N/A | 0 | N/A |

TABLE 5
MEAN INJECTION SITE HISTOLOGY SCORES ON DAY 84 (Example 5)

| | Immunogen | IL-2 | Immunogen | IL-2 | Immunogen | IL-2 |
|---|---|---|---|---|---|---|
| Group # | Site 1 | Site 2 | Site 3 | Site 4 | Site 5 | Site 6 |
| 1. 100 μg GnRHDT in ISA 703 (inj. 1) emulsion | 1.0 | N/A | 0.3 | N/A | 0.5 | N/A |
| 1.5 mg GnRHDT (inj. 2&3)MLV | | | | | | |
| 2. 1.5 mg GnRHDT, 0 cu IL-2 MLV i.m. | 0.5 | 0.0 | 0.5 | 0.5 | 0.3 | 0.0 |
| 3. 1.5 mg GnRHDT, 1,000 cu IL-2 MLV; i.m. | 0.3 | 0.5 | 0.3 | 0.5 | 1.0 | 0.3 |
| 4. 1.5 mg GnRHDT, 10,000 cu IL-2 MLV; i.m. | 0.5 | 0.5 | 1.0 | 0.3 | 0.5 | 0.3 |
| 5. 1.5 mg GnRHDT, 100,000 cu IL-2 MLV; i.m. | 0.5 | 0.3 | 0.5 | 0.3 | 1.3 | 0.3 |
| 6. 3 mg GnRHDT, 0 cu IL-2 MLV; i.m. | 0.3 | 1.5 | 0.8 | 0.5 | 1.8 | 0.0 |
| 7. 3 mg GnRHDT, 1,000 cu IL-2 MLV; i.m. | 0.8 | 0.5 | 0.5 | 0.3 | 0.5 | 0.0 |
| 8. 3 mg GnRHDT, 10,000 cu IL-2 MLV; i.m. | 0.0 | 0.3 | 0.5 | 0.0 | 0.5 | 0.0 |
| 9. 3 mg GnRHDT, 100,000 cu IL-2 MLV; i.m. | 0.5 | 1.3 | 1.0 | 0.3 | 1.0 | 0.0 |
| 10. 1.5 mg GnRHDT in PBS solution i.m. | 0.0 | N/A | 0.0 | N/A | 0.0 | N/A |
| 11. 3 mg GnRHDT in PBS solution i.m. | 0.0 | N/A | 0.0 | N/A | 0.3 | N/A |
| 12. 3 mg GnRHDT, 100,000 cu IL-2 MLV; s.c. | 1.0 | 1.0 | 1.3 | 1.3 | 2.0 | 0.5 |
| 13. 100 μg GnRHDT in ISA 703 emulsion, i.m. | 0.8 | N/A | 1.0 | N/A | 2.8 | N/A |

**Contains moderate to marked calcification
Histopathology Scoring
0-0.5: No inflammation or other histopathological abnormality.
1.0-1.5: Mild active or residual chronic inflammation.
2.0-2.5: Moderate active or chronic inflammation.
3.0: Severe chronic or active inflammation.

### EXAMPLE 6

### G17DT-Liposome Optimal Lipid:Protein Ratio and Hydration Solution

As shown in foregoing Example 3, high doses of conjugate are effective when encapsulated in liposomes. To further optimize the G17DT liposome immunogen, we performed the experiment described in this example wherein G17DT liposomes were formulated at different lipid:protein ratios in order to establish an optimal lipid:protein ratio. Furthermore, we tested two hydration solutions, including water and water containing 5% ethanol to determine their effect upon immunogenicity and injection site reactogenicity.

Thus, the present example evaluated the immunogenicity and local tolerance values of high doses of hG17DT (either 1.5, 3.0 or 4.5 mg) formulated as the previously described DMPC liposomes but at lipid:protein ratios of 50:1, 100:1, 150:1 and 300:1. The efficacy of the formulations were compared with a Montanide® ISA 703 emulsion containing G17DT conjugate (100 μg dose in a 0.2 ml emulsion volume), as controls.

Hydration of liposomes is the final step in preparation of the injectable formulation. Typically, hydration solution is added in a series of aliquots to lyophilized lipid (the protein can be with the lyophilized lipid or in the hydration solution) with vortex mixing after each addition of hydration media. Generally, water for injection (WFI) is used. Here, we also tested WFI that was supplemented with 5% ethanol (EtOH), which has the added advantage of enhancing hydration of the liposomes.

Specifically, eight rabbit groups (n=6 per group) were immunized with the G17DT immunogens encapsulated in liposomes. The liposomes were injected intramuscularly (i.m.) with 1.0 ml dose volumes, given as 2 injections of 0.5 mL each on each injection day. (Groups 1-7). The animals of Group 8 received 100 μg G17DT in Montanide ISA 703 in 0.2 mL intramuscularly. The injections were administered in a series of three sets of injections, given on days 0, 28 and 56. Serum samples were collected at 14-day intervals over the 84 days of treatment at which and all rabbits were euthanized and scored for injection site reactions. Biopsies from two animals per group were evaluated by microscopic examination.

Anti-G17 antibody responses were measured by ELISA, a direct binding assay method, wherein antibody binding to wells coated with gastrin target antigen was detected indirectly by using an anti-antibody-enzyme complex plus enzyme substrate.

### Experimental Procedure

G17DT Immunogen Formulations

The test materials consisted of various formulations of G17DT Immunogen, which were prepared from the following components.

1. hG17DT; hG17 (1-9) pGlu-Gly-Pro-Trp-Leu-Glu-Glu-Glu-Glu-Ser-Ser-Pro-Pro-Pro-Pro-Cys coupled to an immunogenic carrier. (SEQ ID NO: 18 in the Sequence Listing);

2. Phosphate Buffered Saline (PBS): [0.017M Na₂HPO₄+0.001M KH₂PO₄+0.14M NaCl, pH 7.2];

3. Montanide®ISA 703: (Seppic; Paris, France);

4. DMPC: hG17DT Liposomes;

7. Water for Injection (WFI)

8. WFI containing 5% Ethanol by volume (WFI/5% EtOH).

The hG17DT immunogen was prepared in accordance with methods disclosed in U.S. Pat. No. 5,468,494, which methods have been incorporated herein by reference.

Test Formulations

The G17DT Immunogens were aseptically formulated in the combinations shown in Table I. For all liposome formulations, the appropriate volume of sterile WFI or WFI/5% EtOH was added into each vial in 100 μl increments with vigorous vortexing between additions. The ISA 703 emulsion was prepared using a standard hand-mixing method using a 70:30 (oil:aqueous phase, wt:wt) ratio. PBS was used as diluent to prepare the aqueous phase. The test materials were dispensed into syringes and stored under refrigeration (2-8° C.).

In Vivo Protocol:

Adult, virgin female, pathogen-free New Zealand white rabbits were used in the study. The rabbits were grouped (n=6) and immunized with the G17DT immunogens as shown in Table I. The total dose volume of 1.0 mL per injection was split into two injections of 0.5 mL each on each of the three injection days. The rabbits received three immunizations, on days 0, 28, and 56. The injections were intramuscular (i.m.), and were given in the hind legs (0.5 mL into each leg) following a standard protocol. The injection sites were tattooed for later identification. Control rabbits immunized with the emulsified Montanide ISA 703 G17DT immunogen received one 0.2 mL dose of immunogen on each of the injection days, given in the rear legs, alternating right-left-right on days 0-28-56.

To assess immunogenicity, sera were prepared from blood samples obtained from each rabbit every 14 days until day 84, when the rabbits were euthanized. Blood (15 ml per bleed) was collected from marginal ear veins using an 18 gauge needle, then stored at 2-8° C. overnight to allow for clot shrinkage. The samples were then centrifuged (400×g) and the sera were removed by pipette and frozen as individual samples at −10 to −25° C. until assayed.

Antibody Assay:

Anti-Gastrin antibody titers were measured in the sera samples by ELISA; the data is presented in Table II. Sera tested for antibodies were collected on test days 0, 14, 28, 42, 56, 70, and 84.

Gross Pathology:

All the test animals were examined for gross injection site pathology on day 84. Injection sites were located by tattoos, the skin was reflected to fully expose the muscle, and a transverse incision was made completely through the muscle at each injection site. Tissues were visually evaluated for gross pathology on a scale of 0-3, where a score of 0 indicated that the tissue appeared normal, and a score of 3 indicated the presence of an extensive inflammatory reaction throughout the injection area of the tissue. Scores of 1 and 2 represent intermediate levels of local reaction. Individual gross pathology scores of Example 6 are given in Table III.

Microscopic Pathology Observations

After grading for gross pathology, two rabbits per treatment group were randomly selected for microscopic pathology observation. The i.m. injection sites were biopsied by excising a 2 to 2.5 cm length of quadriceps muscle with a scalpel and immediately submerging the tissue specimens in a minimum volume of 25 ml of buffered formalin. Each sample was placed in a separate vial and allowed to fix in the formalin for a minimum of 24 hours. The vials were processed for histopathological evaluation of a region of the biopsy for microscopic examination, after paraffin embedding, sectioning at 5 μm thickness, mounting, and H and E staining. Individual histology scores and the scoring system of Example 6 are given in Table IV.

Macroscopic Pathology Observations

The formulations of the present invention exhibit low reactogenicity in a mammal, particularly a rabbit and most particularly in a human. The rabbit is the best available model for human reactogenicity and immunogenicity. An additional advantage of the rabbit model is that it generally exhibits a reactogenicity similar to or even higher than that of human subjects.

The key indicator of immunogen-induced, injection site reactogenicity is the extent of abnormality throughout the injected muscle as assessed by visual inspection. Thus, the acceptability of a formulation is determined in terms of its capacity to induce muscle reactions judged solely on the basis of the gross reaction scores assigned upon visual examination of the injected tissue.

Low reactogenicity as used herein corresponds to a finding of minimum pathology at the injection site, i.e. either no visible pathology, or a gross appearance score of 1.0 or less. A gross appearance score of 1.0 corresponds to minimum pathology (see Appendix: Evaluation of Injection site Reactions).

By the scoring system employed in these studies, visual (macroscopic) pathology scores of 1 or less are considered to be clinically very acceptable; whereas, scores in excess of 1 are less acceptable. High scores, of 2.5 to 3, are poorly acceptable. Moreover, visual scores of 0 to 0.5 are considered to be essentially normal tissue (i.e., non-pathologic).

Statistical Analysis:

Both the mean and median anti-Gastrin titers were calculated (Table II) from the individual antibody titer and group responses. Peak mean antibody titers were compared between groups, using Student's t test (p<0.05).

Mean injection site reaction scores were calculated from the gross pathology observations. Mean gross histology scores were calculated and are given in Tables III and IV, respectively.

Immunologic Results:

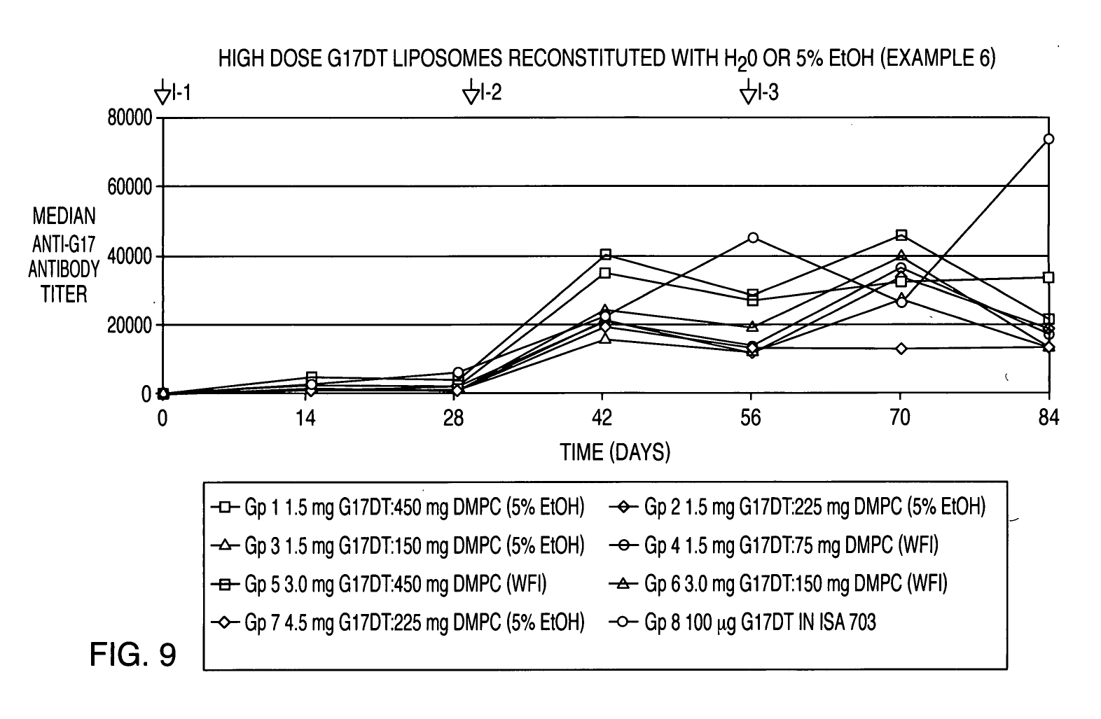

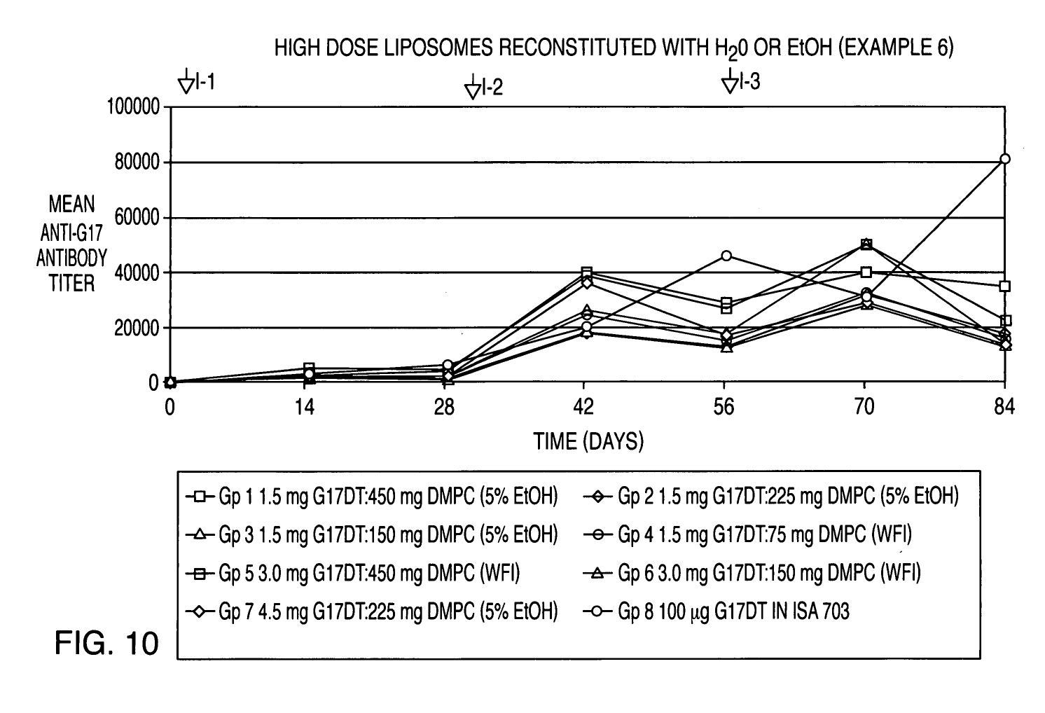

The anti-hG17 antibody responses generated by each group over the course of the 84 day immunogenicity test in vivo were measured by ELISA. Individual, mean and median antibody titers are given in Table B. The mean titers are plotted in FIG. 9, with the median titer plots shown in FIG. 10.

As shown in the drawings (FIG. 9 and FIG. 10), the control G17DT immunogen emulsion, formulated in Montanide® ISA 703 and delivering 100 μg G17DT/dose (Group 8), induced responses that were similar to those induced by the liposome formulations until day 84, when the ISA 703 responses were elevated to about twice those of the liposomes. In comparison, the responses of rabbits injected i.m. with liposome preparations were lower in titer and tended to present shorter, more highly defined booster responses after injections #2 and #3. However, all liposome formulations induced and sustained titers in excess of 10,000. The responses of Group1 (1.5 mg G17DT, 450 mg DMPC, hydrated in WFI/5% EtOH) were particularly stable in terms of sustained antibody production over the course of the study from days 42 through 84. This particular formulation, at a 1:300 ratio (wt:wt) of lipid:protein, which was hydrated in WFI/5% EtOH, was especially effective.

There was no significant difference between the peak mean response of Group 8 in comparison with the peak responses of the liposome groups, with the exception of that of group 4 (p=0.096), indicating that the liposome based immunogens were effective. Liposomes prepared by hydration with WFI and WFI/5% EtOH were particularly effective. It was noted that the viscosity of the liposomes hydrated with WFI/5% EtOH was elevated, which may increase their effectiveness for long term immunization where a stable level of antibody production over time is desirable. The antibody response of Group 1 provides and example of such a response.

The injection site reaction grades were assessed visually in all rabbits on day 84. As the data show, injection site reactions were minimal for all groups except Group 8 (standard emulsion preparation). This subject group presented scores >1 in 2 of 6 animals at the third immunogen injection site. In Groups 1-7, no reaction scores in excess of 0.5 were observed in 252 sites. Thus, visual assessment indicated that the liposome preparations were very well tolerated when administered i.m.

Microscopic Pathology Observations

Histological examination of injection-site tissues is conducted to produce data that support the macroscopic pathologic findings with histopathology descriptions of the nature of the inflammatory response. Because the histopathology focuses at the cellular level and not upon the degree of overall inflammation in the injected tissue, histopathology is not used to pass judgment upon the overall acceptability, or lack of such, of a vaccine formulation in terms of injection site tolerability.

The histopathology results on day 84 are shown in Table IV. Microscopic pathology readings of the injection site biopsies were generally in accord with the gross visual evaluation results, with the highest scores occurring at sites of the third injection of immunogen formulated in ISA 703. Inflammatory reactions were minimal for nearly all of the liposome i.m. injection sites. The muscle reaction scores seen in Group 8 are typical for water-in-oil emulsions. It should be noted that the visual and histologic reaction grading systems are independent and not correlated against one another. Generally, the histology reaction scores exceed the visual scores. Sites receiving scores of 0.5 or less are considered to have no pathology. Nevertheless, significantly less muscle inflammation was induced by the liposomes than the water-in-oil emulsions.

Conclusion:

The results of the experiment of Example 6 demonstrate that liposomes formulated at a lipid-to-protein ratio of 300:1, delivering 1.5 mg G17DT and hydrated with a solution of 5% EtOH in WFI, induced sustained levels of anti-G17 antibodies at acceptable titers. Similarly, the other liposome formulations tested herein induced comparable levels of anti-G17 antibody, though the response levels were either not quite as high (though not statistically significantly lower) or not as steady state as the aforementioned group. Nevertheless, very low tissue reactogenicity was observed for all of the liposome formulations, in comparison with the Montanide ISA 703 emulsion control. The low levels of injection site reactions indicate that increased injection frequencies would likely be acceptable as a means of increasing the response levels while maintaining minimal injection site reactions. These results indicate that the high protein liposomal preparations of G17DT can be optimized by selection of effective lipid:protein ratios as well as by inclusion of ethanol to 5% in the liposome hydration medium.

TABLE I
(Example 6)
IMMUNOGEN FORMULATIONS

| | | | | Protein/ | | |
|---|---|---|---|---|---|---|
| Immuno- | | | | Lipid | | |
| gen | | | Conjugate | Ratio | Hydrate | Rabbit |
| Lot No. | Vehicle | DMPC | G17DT | (w/w) | Solution | Group |
| 2A | liposomes | 450 mg | 1.5 mg | 1:300 | 5% | 1 |
| | | | | | EtOH | |
| 2B | liposomes | 225 mg | 1.5 mg | 1:150 | 5% | 2 |
| | | | | | EtOH | |
| 2C | liposomes | 150 mg | 1.5 mg | 1:100 | 5% | 3 |
| | | | | | EtOH | |
| 2D | liposomes | 75 mg | 1.5 mg | 1:50 | WFI | 4 |
| 2E | liposomes | 450 mg | 3.0 mg | 1:150 | WFI | 5 |
| 2F | liposomes | 150 mg | 3.0 mg | 1:50 | WFI | 6 |
| 2G | liposomes | 225 mg | 4.5 mg | 1:50 | 5% | 7 |
| | | | | | EtOH | |
| 2H | Montanide | — | 100 μg | — | — | 8 |
| | ISA 703 | | | | | |

TABLE II
(Example 6)
RABBIT ANTI-G17 ANTIBODY RESPONSES

| | | Inj. 1 | | | | | | |
|---|---|---|---|---|---|---|---|---|
| | | Pre- | | Inj. 2 | | Inj. 3 | | Euthanize |
| | | bleed | Bleed 1 | Bleed 2 | Bleed 3 | Bleed 4 | Bleed 5 | Bleed 6 |
| | | Day | Day | Day | Day | Day | Day | Day |
| Group | Rabbit # | 0 | 14 | 28 | 42 | 56 | 70 | 84 |
| Gp 1 | Mean | 0 | 2,751 | 3,703 | 40,033 | 29,333 | 40,183 | 35,567 |
| 1.5 mg G17DT: 450 mg | Median | 0 | 2,497 | 2,132 | 35,200 | 27,500 | 32,800 | 33,900 |
| DMPC | S.D. | Pool | 1,887 | 4,195 | 19,895 | 11,904 | 15,547 | 12,645 |
| (5% EtOH) | | | | | | | | |
| Gp 2 | Mean | 0 | 1,421 | 1,311 | 17,900 | 13,097 | 32,550 | 18,065 |
| 1.5 mg G17DT: 225 mg | Median | 0 | 1,123 | 1,172 | 19,450 | 11,892 | 34,350 | 18,950 |
| DMPC | S.D. | Pool | 1,028 | 876 | 5,723 | 6,307 | 14,008 | 7,781 |
| (5% EtOH) | | | | | | | | |
| Gp 3 | Mean | 0 | 1,276 | 764 | 18,717 | 13,042 | 29,000 | 13,925 |
| 1.5 mg G17DT: 150 mg | Median | 0 | 1,228 | 773 | 16,350 | 12,800 | 28,100 | 13,600 |
| DMPC | S.D. | Pool | 693 | 240 | 6,812 | 4,403 | 14,221 | 4,140 |
| (5% EtOH) | | | | | | | | |
| Gp 4 | Mean | 0 | 1,933 | 1,564 | 24,800 | 15,222 | 32,883 | 16,268 |
| 1.5 mg G17DT: 75 mg | Median | 0 | 1,168 | 828 | 20,900 | 13,800 | 36,650 | 17,500 |
| DMPC | S.D. | Pool | 1,655 | 1,365 | 12,522 | 6,826 | 13,489 | 7,332 |
| (WFI) | | | | | | | | |
| GP 5 | Mean | 0 | 5,350 | 4,943 | 38,850 | 27,100 | 50,183 | 22,826 |
| 1.5 mg G17DT: 450 mg | Median | 0 | 5,059 | 4,162 | 40,500 | 28,850 | 46,050 | 21,850 |
| DMPC | S.D. | Pool | 2,437 | 2,920 | 13,942 | 5,881 | 26,085 | 10,494 |
| (WFI) | | | | | | | | |
| Gp 6 | Mean | 0 | 1,753 | 985 | 26,517 | 18,457 | 51,150 | 14,116 |
| 1.5 mg G17DT: 150 mg | Median | 0 | 1,694 | 911 | 24,450 | 19,600 | 40,300 | 14,150 |
| DMPC | S.D. | Pool | 135 | 338 | 8,935 | 7,062 | 40,314 | 5,464 |
| (WFI) | | | | | | | | |
| Gp 7 | Mean | 0 | 1,514 | 987 | 29,867 | 18,017 | 37,600 | 15,344 |
| 1.5 mg G17DT: 225 mg | Median | 0 | 1,525 | 729 | 24,400 | 15,150 | 35,250 | 13,900 |
| DMPC | S.D. | Pool | 514 | 586 | 15,115 | 10,057 | 17,738 | 6,708 |
| (5% EtOH) | | | | | | | | |
| Gp 8 | Mean | 0 | 3,205 | 6,301 | 20,346 | 46,267 | 31,400 | 81,433 |
| 1.5 mg G17DT in ISA | Median | 0 | 2,813 | 6,256 | 21,950 | 45,350 | 26,800 | 73,850 |
| 703 | S.D. | Pool | 2,000 | 3,550 | 7,939 | 23,697 | 15,253 | 63,302 |

TABLE III
(Example 6)
INJECTION SITE REACTIONS ON DAY 84

| Group | Rabbit # | Site 1A | Site 1B | Site 2A | Site 2B | Site 3A | Site 3B |
|---|---|---|---|---|---|---|---|
| Gp 1 | Mean | 0.1 | 0.1 | 0.3 | 0.3 | 0.3 | 0.3 |
| 1.5 mg G17DT: 450 mg | No. >1 | 0.0 | 0.0 | 0.0 | 0.0 | 0.0 | 0.0 |
| DMPC | | | | | | | |
| (5% EtOH) | | | | | | | |
| Gp 2 | Mean | 0.2 | 0.2 | 0.3 | 0.1 | 0.3 | 0.3 |
| 1.5 mg G17DT: 225 mg | No. >1 | 0.0 | 0.0 | 0.0 | 0.0 | 0.0 | 0.0 |
| DMPC | | | | | | | |
| (5% EtOH) | | | | | | | |
| Gp 3 | Mean | 0.3 | 0.1 | 0.1 | 0.1 | 0.3 | 0.4 |
| 1.5 mg G17DT: 150 mg | No. >1 | 0.0 | 0.0 | 0.0 | 0.0 | 0.0 | 0.0 |
| DMPC | | | | | | | |
| (5% EtOH) | | | | | | | |
| Gp 4 | Mean | 0.0 | 0.0 | 0.0 | 0.0 | 0.0 | 0.0 |
| 1.5 mg G17DT: 75 mg | No. >1 | 0.0 | 0.0 | 0.0 | 0.0 | 0.0 | 0.0 |
| DMPC | | | | | | | |
| (WFI) | | | | | | | |
| Gp 5 | Mean | 0.0 | 0.1 | 0.4 | 0.3 | 0.3 | 0.3 |
| 1.5 mg G17DT: 450 mg | No. >1 | 0.0 | 0.0 | 0.0 | 0.0 | 0.0 | 0.0 |
| DMPC | | | | | | | |
| (WFI) | | | | | | | |
| Gp 6 | Mean | 0.3 | 0.2 | 0.1 | 0.1 | 0.1 | 0.2 |
| 1.5 mg G17DT: 150 mg | No. >1 | 0.0 | 0.0 | 0.0 | 0.0 | 0.0 | 0.0 |
| DMPC | | | | | | | |
| (WFI) | | | | | | | |
| Gp 7 | Mean | 0.0 | 0.0 | 0.1 | 0.1 | 0.2 | 0.2 |
| 1.5 mg G17DT: 225 mg | No. >1 | 0.0 | 0.0 | 0.0 | 0.0 | 0.0 | 0.0 |
| DMPC | | | | | | | |
| (5% EtOH) | | | | | | | |
| Gp 8 | Mean | 0.3 | Na | 0.7 | na | 1.3 | na |
| 1.5 mg G17DT in ISA | No. >1 | 0.0 | Na | 0.0 | na | 2.0 | na |
| 703 | | | | | | | |

TABLE IV
(Example 6)
INDIVIDUAL AND MEAN INJECTION SITE HISTOLOGY SCORES ON DAY 84

| Group # | Rabbit # | Site 1A | Site 1B | Site 2A | Site 2B | Site 3A | Site 3B |
|---|---|---|---|---|---|---|---|
| Gp 1 | Mean | 0.0 | 0.3 | 1.3 | 0.5 | 1.0 | 0.5 |
| 1.5 mg G17DT: 450 mg | | | | | | | |
| DMPC (5% EtOH) | | | | | | | |
| Gp 2 | Mean | 0.3 | 0.5 | 0.8 | 0.5 | 0.8 | 0.5 |
| 1.5 mg G17DT: 450 mg | | | | | | | |
| DMPC (5% EtOH) | | | | | | | |
| Gp 3 | Mean | 0.5 | 0.5 | 0.5 | 0.5 | 0.5 | 0.5 |
| 1.5 mg G17DT: 450 mg | | | | | | | |
| DMPC (5% EtOH) | | | | | | | |
| Gp 4 | Mean | 0.5 | 0.5 | 0.8 | 0.5 | 0.5 | 0.3 |
| 1.5 mg G17DT: 450 mg | | | | | | | |
| DMPC (5% EtOH) | | | | | | | |
| Gp 5 | Mean | 0.3 | 0.3 | 0.5 | 0.5 | 0.5 | 0.5 |
| 1.5 mg G17DT: 450 mg | | | | | | | |
| DMPC (5% EtOH) | | | | | | | |
| Gp 6 | Mean | 0.3 | 0.5 | 0.3 | 0.5 | 1.0 | 1.0 |
| 1.5 mg G17DT: 450 mg | | | | | | | |
| DMPC (5% EtOH) | | | | | | | |
| Gp 7 | Mean | 0.3 | 0.5 | 0.5 | 0.3 | 0.5 | 0.5 |
| 1.5 mg G17DT: 450 mg | | | | | | | |
| DMPC (5% EtOH) | | | | | | | |
| Gp 8 | Mean | 0.8 | na | 1.3 | Na | 2.5 | na |
| 1.5 mg G17DT: 450 mg | | | | | | | |
| DMPC (5% EtOH) | | | | | | | |

Histopathology Scoring
0-0.5: No inflammation or other histopathological abnormality.
1.0-1.5: Mild active or residual chronic inflammation.
2.0-2.5: Moderate active or chronic inflammation.
3.0: Severe chronic or active inflammation

### Appendix Evaluation of Injection Site Reactions

### Protocol: Gross Evaluation of Injection Sites in Rabbit Thigh Muscles

Purpose: evaluate the gross (macroscopic) appearance of the thigh muscle after injection of test materials.

Procedure: The skin of the euthanized animal is peeled off of the thigh by making a transverse and a longitudinal incision and then peeling off the skin. Care is taken to make a clean separation from muscle tissue, without damaging the latter.

The injection site(s) are marked by tattoo at the time injection is given. If two injections are given in a single muscle, the sites should be about 4 cm apart.

Using a sharp lancet, each injection site is incised to expose the interior of the thigh muscle. Additional incisions can be made to ensure complete viewing and assessment of pathology. Biopsy specimens are preserved in HistoChoice™.

Sampling: Some animals in each treatment group may undergo biopsy for further histological evaluation. Biopsies should be extensive enough to allow full evaluation of any pathology.

If a subsequent animal(s) shows gross features that are either not seen in the index animal, or merit histological examination for any other reason, biopsy(ies) is taken.

Scale for evaluation of gross appearance

0—Normal tissue.

No visible pathology. At times yellow fatty/fibrous tissue appears after complete resolution of inflammation in the muscle tissue. Such a change is not rated as pathological.

1—Minimal pathology.

A typical appearance includes small, (<3 mm in diameter), hard nodules, representing encapsulated and resolving sterile abscesses or inflammatory sites. The combined volume of such lesions is less than 5% of the total thigh muscle volume.

2—Moderate pathology.

Nodules are larger (3-10 mm in diameter). They can be hard to the touch (old fibrosis) or soft (more recently encapsulated). On squeezing such lesions, pus or injection material may be expressed. Free (unencapsulated) material may occasionally be seen. In that case, its longitudinal diameter is no larger than 10 mm. The combined volume of the lesions is between 5-10% of the total thigh muscle volume.

3—Severe pathology.

Large, encapsulated or unencapsulated lesions, larger than 10 mm in longitudinal diameter. Typically, lesions contain pus (sterile abscesses) or injection material (emulsion). Total volume of lesions >10% of thigh muscle volume.

Intermediate grades

When lesions don't fall unequivocally within the definition of a certain grade, intermediate grades are assigned, e.g., 0.5, 1.5 or 2.5.

Epilogue

The aforegoing examples illustrate, but by no means limit, advantageous aspects of this liposomal delivery system having the high ratio of lipid to encapsulated water-soluble substance. It is clear that the experienced practitioner of the invention would be able to apply this system to other useful substances in the treatment of human diseases or disorder, such as the delivery of water-soluble factors, cofactors, hormones, analogues or modifications thereof.

We claim:

1. An injectable liposomal composition for delivery of a water-soluble substance, the composition comprising:
a plurality of liposomal vesicles comprising a high weight ratio of a lipid to an encapsulated water-soluble substance so as to achieve a high efficiency of encapsulation.

2. The composition of claim 1, wherein the encapsulation efficiency is more than about 50%.

3. The composition of claim 1, wherein the encapsulation efficiency is more than about 80%.

4. The composition of claim 1, wherein the liposomal vesicles are multilamellar vesicles (MLV).

5. The composition of claim 1, wherein the water-soluble substance comprises more than one compound.

6. The composition of claim 1, wherein the water-soluble substance is selected from the group consisting of a protein, a proteoglycan and a carbohydrate.

7. The composition of claim 1, wherein the water-soluble substance comprises a vaccine.

8. The composition of claim 7, wherein the vaccine is directed against a hormone or a hormone cognate receptor.

9. The composition of claim 7, wherein the vaccine comprises at least one hormone-immunomimic peptide or at least one hormone receptor-immunomimic peptide, and wherein the immunomimic peptide is conjugated to an immunogenic hydrophilic carrier protein.

10. The composition of claim 1, wherein the weight ratio of lipid to encapsulated substance ranges from about 50 to about 1000.

11. The composition of claim 1, wherein the weight ratio of lipid to encapsulated substance is about 300.

12. The composition of claim 9, wherein the immunomimic peptide is a synthetic sequence selected from the group consisting of gastrin G-17, gastrin G-34, GnRH, hCG and fragments thereof.

13. The composition of claim 12, wherein the synthetic sequence is the gastrin G-17 of SEQ NO:1.

14. The composition of claim 13, wherein the synthetic gastrin G-17 fragment sequence is a sequence selected from the group consisting of SEQ ID NOS:3-8.

15. The composition of claim 12, wherein the synthetic sequence is the G-34 peptide of SEQ ID NO:12.

16. The composition of claim 12, wherein the synthetic peptide is the GnRH immunomimic peptide of SEQ ID NO:15.

17. The composition of claim 12, wherein the synthetic peptide is the hCG immunomimic peptide of SEQ ID NO:16.

18. The composition of claim 1, wherein the lipid comprises a hydrophobic chain and a polar or chemically reactive portion.

19. The composition of claim 1, wherein the lipid comprises a hydrocarbon chain or steroid tail group, and a polar head group.

20. The composition of claim 18, wherein the polar head group or chemically reactive portion comprises an acid, alcohol, aldehyde, amine or ester group.

21. The composition of claim 1, wherein the lipid comprises a phospholipid.

22. The composition of claim 21, wherein the phospholipid is selected from the group consisting of phosphatidic acid, phosphatidyl choline, phosphatidyl ethanolamine, phosphatidyl glycerol, phosphatidyl inositol, and sphingomyelin.

23. The composition of claim 1, wherein the liposome comprises at least about 70 mole percent dimyristoyl phosphatidylcholine (DMPC).

24. The composition of claim 7, wherein the encapsulated vaccine has a dose of from about 50 μg to about 5 mg.

25. The composition of claim 8, wherein the encapsulated anti-hormone vaccine or anti-hormone receptor vaccine has a dose ranging from about 0.3 mg to about 5 mg.

26. The composition of claim 9, wherein the immunomimic peptide is conjugated to the immunogenic carrier through a spacer peptide.

27. The composition of claim 26, wherein the spacer peptide is selected from the group consisting of SEQ NOS: 9, 10, and 11.

28. The composition according to claim 1, wherein the liposome vesicles encapsulate a water-soluble immunogen and a water-soluble high molecular weight immunomodulatory substance, either together or in separate liposome vesicles.

29. The composition according to claim 1, wherein the liposome vesicles encapsulate a water-soluble immunogen and a water-soluble low molecular weight immunomodulatory substance, either together or in separate liposome vesicles.

30. The composition according to claim 28, wherein the high molecular weight immunomodulatory substance comprises a cytokine.

31. The composition according to claim 30, wherein the low molecular weight substance is selected from the group consisting of nor MDP, threonyl MDP, murabutide, N-acetylglucosaminyl-MDP, and murametide.

32. An aseptic composition comprising an injectable aqueous suspension of the composition of any one of claims 7-17.

33. A pharmaceutical formulation comprising a therapeutically effective amount of the composition of claim 1, and a pharmaceutically acceptable carrier.

34. A method of treatment of a disorder or disease, comprising administering to a patient in need of the treatment a therapeutically effective amount of the pharmaceutical formulation of claim 33.

35. A method for producing a liposomal vaccine comprising the steps of: preparing phospholipid multilamellar vesicles and encapsulating a water-soluble immunogen or an immunomodulating substance, or both, wherein the liposomes have a high lipid:protein ratio.

36. The method of claim 35 wherein the lipid:protein ratio is in the range from about 50 to about 1000.

37. The method of claim 36 wherein the ratio is about 500.

38. The method of claim 37 wherein the ratio is about 300.

39. A liposomal composition of high lipid:protein weight ratio comprising an immunogenic construct of immunogenic carrier conjugated to peptide selected from the group consisting of SEQ ID NOS: 17, 18, 19, and 20.

40. A method for producing a liposomal vaccine containing a high dose of immunogen, the method comprising:
rehydrating a lyophilized lipid complement with water or an aqueous ethanol solution, at which step the immunogen is contained either in the lipid complement or the aqueous ethanol solution.

41. The composition of claim 1, wherein the composition exhibits low injection site reactogenicity in a mammal.

42. The composition of claim 41, wherein the mammal is a rabbit.

43. The composition of claim 42, wherein the injection site reactogenicity is substantially no inflammation or other gross pathological abnormality.

44. The composition of claim 41, wherein the mammal is a human.

45. The composition of claim 44, wherein the injection site reactogenicity is substantially no inflammation or other gross pathological abnormality.

46. The composition of claim 41, wherein the low reactogenicity is exhibited when the composition is delivered intramuscularly.

47. The composition of claim 41, wherein the low reactogenicity is exhibited when the composition is delivered subcutaneously.

48. The composition of claim 41, wherein the low reactogenicity is exhibited when the composition is delivered intradermally.

49. A method of treatment according to claim 34, wherein the composition is delivered intramuscularly.

50. A method of treatment according to claim 34, wherein the composition is delivered subcutaneously.

51. A method of treatment according to claim 34, wherein the composition is delivered intradermally.


SEQUENCE LISTING (20 sequences)

SEQ ID NO: 1 - protein, 17 aa, Homo sapiens
  MOD_RES (1)..(1): PYRROLIDONE CARBOXYLIC ACID
EGPWLEEEEEAYGWMDF

SEQ ID NO: 2 - protein, 18 aa, Homo sapiens
  MOD_RES (1)..(1): PYRROLIDONE CARBOXYLIC ACID
EGPWLEEEEEAYGWMDFG

SEQ ID NO: 3 - protein, 5 aa, Artificial
  synthetic
EGPWL

SEQ ID NO: 4 - protein, 6 aa, artificial
  synthetic
EGPWLE

SEQ ID NO: 5 - protein, 7 aa, Artificial
  synthetic
EGPWLEE

SEQ ID NO: 6 - protein, 8 aa, Artificial
  synthetic peptide of amino acid sequence 1-8 of human gastrin 17 linked to spacer peptide
EGPWLEEE

SEQ ID NO: 7 - protein, 9 aa, Artificial
  synthetic
EGPWLEEEE

SEQ ID NO: 8 - protein, 10 aa, Artificial
  synthetic
EGPWLEEEEE

SEQ ID NO: 9 - protein, 6 aa, Artificial
  synthetic peptide spacer
RPPPPC

SEQ ID NO: 10 - protein, 7 aa, Artificial
  synthetic peptide spacer
SSPPPPC

SEQ ID NO: 11 - protein, 8 aa, Artificial
  synthetic peptide spacer
SPPPPPPC

SEQ ID NO: 12 - protein, 22 aa, Artificial
  synthetic peptide of amino acid sequence 1-6 of human gastrin 34 linked to a spacer peptide
ELGPEGPPHLVADPSKKEGPWL

SEQ ID NO: 13 - protein, 13 aa, Artificial
  synthetic peptide of amino acid sequence 1-6 of human gastrin 34 linked to a spacer peptide
ELGPEGSSPPPPC

SEQ ID NO: 14 - protein, 13 aa, Homo sapiens
CPPPPSSELGPEG

SEQ ID NO: 15 - protein, 9 aa, Artificial
  synthetic
EHWSYGLRP

SEQ ID NO: 16 - protein, 35 aa, Artificial
  synthetic peptide of amino acid sequence 138-145 of human chorionic gonadotrophin hormone linked to a specer peptide
DDPRTEDSSSSKAPPPSLPSPSRLPGPSDTPILPQ

SEQ ID NO: 17 - protein, 16 aa, Artificial
  synthetic peptide of amino acid sequence of human gastrin 17 linked to a spacer peptide
ESDTPIPQSPPPPPPC

SEQ ID NO: 18 - protein, 17 aa, Artificial
  synthetic peptide of GnRH amino acid sequence linked to a spacer peptide
EGPWLEEEEESSPPPPC

SEQ ID NO: 19 - protein, 17 aa, Artificial
  synthetic peptide of amino acid sequence 138-145 of human chorionic gonadotrophin hormone linked to a spacer peptide
CPPPPSSEHWSYGLRPG

SEQ ID NO: 20 - protein, 15 aa, Artificial
  synthetic peptide of a spacer peptide linked to amino acid sequence 138-145 of human chorionic gonadotrophin
CPPPPSSSDTPILPQ